SEQUENCE LISTING (17 sequences)

SEQ ID NO: 1 - DNA, 22 bp, artificial
  synthetic sequence
tgggaaccagcctataccccag

SEQ ID NO: 2 - DNA, 24 bp, artificial
  synthetic sequence
cagttgcagaagggccttctgtac

SEQ ID NO: 3 - DNA, 20 bp, artificial
  synthetic sequence
ggaatgcctgtgtctgcttt

SEQ ID NO: 4 - DNA, 20 bp, Artificial
  Synthetic sequence
tgggtcataatgctgttgcc

SEQ ID NO: 5 - DNA, 20 bp, Artificial
  Synthetic sequence
cgccgctgtccttcggtgtc

SEQ ID NO: 6 - DNA, 21 bp, Artificial
  Synthetic sequence
agggctccggcttccacacat

SEQ ID NO: 7 - DNA, 19 bp, Artificial
  Synthetic sequence
gcatgagcgaggtgcactc

SEQ ID NO: 8 - DNA, 21 bp, Artificial
  Synthetic sequence
tctcgcttcaggtcagccttg

SEQ ID NO: 9 - DNA, 21 bp, Artificial
  Synthetic sequence
gctcctcctgagcgcaagtac

SEQ ID NO: 10 - DNA, 23 bp, Artificial
  Synthetic sequence
ggactcgtcatactcctgcttgc

SEQ ID NO: 11 - DNA, 20 bp, Artificial
  Synthetic sequence
gccaagaggaaggccaagtc

SEQ ID NO: 12 - DNA, 19 bp, Artificial
  Synthetic sequence
agggctcgggtttccacac

SEQ ID NO: 13 - DNA, 26 bp, Artificial
  Synthetic sequence
gacacatatgatggccgaggtgatag

SEQ ID NO: 14 - DNA, 24 bp, Artificial
  Synthetic sequence
ggtgatgtcctcgtctgtagcatc

SEQ ID NO: 15 - DNA, 28 bp, Artificial
  Synthetic sequence
cttcacaaatcctccccaagtagctacc

SEQ ID NO: 16 - DNA, 29 bp, Artificial
  Synthetic sequence
ggtttagagtcatcaagcttctgtctgtg

SEQ ID NO: 17 - protein, 5 aa, Artificial
  Synthetic sequence
YRGDS

(19) United States
(12) Patent Application Publication
(10) Pub. No.: US 2015/0337121 A1
(43) Pub. Date: Nov. 26, 2015

(54) MULTIFUNCTIONAL TUNABLE BIOMATERIALS FOR TISSUE ENGINEERING

(71) Applicant: THE JOHNS HOPKINS UNIVERSITY, Baltimore, MD (US)

(72) Inventors: Anirudha Singh, Baltimore, MD (US); Jianan Zhan, Baltimore, MD (US); Jennifer H. Elisseeff, Baltimore, MD (US)

(21) Appl. No.: 14/410,143

(22) PCT Filed: Jun. 26, 2013
(86) PCT No.: PCT/US2013/047857

§ 371 Date: Dec. 22, 2014

Related U.S. Application Data
(60) Provisional application No. 61/664,355, filed on Jun. 26, 2012.
(60) Provisional application No. 61/720,654, filed on Oct. 31, 2012.

(51) Int. Cl. C08L 5/16; C08L 71/00; C12N 5/00; C08L 33/14

(52) U.S. Cl. CPC C08L 5/16; C08L 33/14; C08L 71/00; C12N 5/0012

(57) ABSTRACT

The present invention provides a multifunctional biomaterial comprising one or more biocompatible polymers and one or more α-cyclodextrin molecules having a plurality of hydroxyl groups capable of being chemically substituted with another functional group or moiety to form a pseudopolyrotaxane structure. The multifunctional biomaterials of the present invention provide synthetic 2D or 3D biomaterial scaffolds and nanofibers that can be decorated with multiple chemical functionalities without altering the base network. The polymer chains can be crosslinked via the terminal ends of the polymers and not through the α-cyclodextrin molecules. The inventive technology is useful for engineering tissue with human stem cells, including, mesenchymal stem cells (hMSCs) and adipose derived stem cells (hADSCs). Methods for making the multifunctional biomaterials and their use in biological application are also provided.

### REFERENCE TO RELATED APPLICATIONS

This application claims the benefit of U.S. Provisional Patent Application Nos. 61/664,355, filed on Jun. 26, 2012, and 61/720,654, filed on Oct. 31, 2012, both of which are hereby incorporated by reference for all purposes as if fully set forth herein.

### INCORPORATION-BY-REFERENCE OF MATERIAL SUBMITTED ELECTRONICALLY

The instant application contains a Sequence Listing which has been submitted in ASCII format via EFS-Web and is hereby incorporated by reference in its entirety. Said ASCII copy, created on Jun. 12, 2013, is named P11696-03_ST25.txt and is 3,353 bytes in size.

### BACKGROUND OF THE INVENTION

Natural extracellular matrix (ECM) is full of chemical signals that modulate the structure and molecular composition of cell-matrix interactions. Any variations in chemical composition of the matrix can change cell-matrix interactions via conformational changes and protein adsorption. These are linked to focal adhesion and ECM production. Depending upon the desired outcome of tissue regeneration or formation, various natural and synthetic polymers are employed for creating biomaterials that can mimic chemical cues of natural ECMs. Hydrogels based on natural polymers, such as alginate, collagen and hyaluronic acid (HA), are widely used for tissue engineering applications; however, these polymers are saturated with specific chemical functionalities, and their chemical compositions play important instructive roles in biological processes (e.g., HA binds cells that have CD44 receptors). Similarly, chemical functionalities (OH, NH₂ and COOH) of some synthetic polymers, such as poly(vinyl alcohol), poly(allyl amine) or poly(acrylic acid), can interact with cells via preferential protein adsorption, leading to specific cellular biological responses. Therefore, the development of a simple but accessible biomaterial design strategy that allows independent modulation of material chemistry, while not interfering inherent composition-dependent specific cell interactions, is needed to understand the independent roles of types and amounts of specific chemical functionalities on different biological outcomes that in the present study are applied to engineering cartilage.

Previous studies have demonstrated that chemical composition of the materials, through chemical functionalities can direct growth and lineage-specific differentiation of MSCs. However, the findings of these studies vary depending on the experimental conditions and chemical composition of the biomaterials. For example, amine-enriched poly (allyl amine) surfaces supported cell adhesion and proliferation, and promoted chondrogenic differentiation of MSCs, while a carboxylic acid-enriched poly(acrylic acid) surface did not promote chondrogenesis. In contrast, others have reported that amine groups containing silane-modified glass surfaces promoted osteogenic differentiation of hMSCs, while OH and COOH promoted chondrogenesis.

Such scaffold biomaterials, including hydrogels and nanofibers, play important roles in dictating cell functions and manipulating tissue development by providing structural support and biophysical and biochemical signals, and transporting nutrients and wastes. An ideal scaffold should have well-defined morphology, sufficient mechanical strength for its intended application and a porous structure that has properties similar to those of the native extracellular matrix (ECM). In this context, scaffolds based on electrospun nanofibers have been studied for tissue engineering applications. These nano- and micro-scale fibers have mechanical strength similar to that of natural tissues and resemble the scale and arrangement of fibrous ECM components, in particular, collagen.

The most widely employed electrospun nanofibrous scaffolds in tissue engineering and drug delivery are based on aliphatic polyesters, such as polycaprolactone (PCL) or polylactide. These materials have a number of useful properties, such as easy processing, biocompatibility and low cost; however, their biological applications are limited because they are hydrophobic and lack active natural cell recognition sites or functional groups along their polyester backbone. An important strategy for polyester functionalization is through copolymerizing polyester with functional monomers prior to polymerization; however, incorporating monomers makes it difficult to obtain high molecular weight polymers for fabricating tissue engineering nanofibrous scaffolds.

There still exists, therefore, a need for novel multifunction biomaterials that can create scaffolds with the desired biological and physical properties for optimizing chondrogenesis of stem cells and tissue engineering.

### SUMMARY OF THE INVENTION

In accordance with an embodiment, the present invention provides a multifunctional biomaterial comprising: one or more biocompatible polymers and one or more α-cyclodextrin molecules having a plurality of hydroxyl groups capable of being chemically substituted with another functional group or moiety; wherein the one or more biocompatible polymers have at least 10 or more monomeric units; and wherein the one or more biocompatible polymers are included in the cavities of the one or more α-cyclodextrin molecules in a skewered manner to obtain a pseudopolyrotaxane configuration.

In accordance with an embodiment, the present invention provides a hydrogel biomaterial comprising one or more poly(ethylene glycol polymers and one or more α-cyclodextrin molecules having a plurality of hydroxyl groups capable of being chemically substituted with another functional group or moiety selected from the group consisting of hydrophobic groups, hydrophilic groups, peptides, C₁-C₆ alkyl, C₂-C₆ alkenyl, C₂-C₆ alkynyl, C₁-C₆ hydroxyalkyl, C₁-C₆ alkoxy, C₁-C₆ alkoxy C₁-C₆ alkyl, C₁-C₆ alkylamino, di-C₁-C₆ alkylamino, C₁-C₆ dialkylamino C₁-C₆ alkyl, C₁-C₆ thioalkyl, C₂-C₆ thioalkenyl, C₂-C₆ thioalkynyl, C₆-C₂₂ aryloxy, C₂-C₆ acyloxy, C₂-C₆ thioacyl, C₁-C₆ amido, C₁-C₆ sulphonamido, C₁-C₆ carboxyl and derivatives, phosphonates and sulfones; wherein the one or more poly(ethylene glycol) polymers have at least 10 or more monomeric units; and wherein the one or more poly(ethylene glycol) polymers are included in the cavities of the one or more α-cyclodextrin molecules in a skewered manner to obtain a pseudopolyrotaxane configuration.

In accordance with an embodiment, the present invention provides a method for making a hydrogel biomaterial comprising: a) obtaining a solution of α-cyclodextrin molecules in a suitable biologically compatible aqueous buffer; b) adding to a) a sufficient amount of hydrophilic polymers or derivatives thereof in a suitable biologically compatible aqueous buffer to create a solution having a polymer concentration of about 1 to about 20% (w/v) and a α-cyclodextrin concentration of about 0.1 to about 10% (w/v); c) mixing the solution of b) for a sufficient time to provide an inclusion step in which hydrophilic polymers or derivatives thereof and cyclodextrin molecules obtain a pseudopolyrotaxane configuration in which the hydrophilic polymers or derivatives thereof are included in the cavity of each of α-cyclodextrin molecule in a skewered manner; d) adding a photoinitiator to the solution of c) to create a final concentration of photoinitiator of between about 0.01 to about 0.1% (w/v); e) exposing the solution of d) to electromagnetic radiation at a wavelength specific to the photoinitiator for a sufficient amount of time to initiate the polymerization of the polymers in the solution; and f) allowing the polymerization to complete.

In accordance with an embodiment, the present invention provides a method for making a 2-dimensional cell-encapsulated hydrogel comprising: a) obtaining a solution of α-cyclodextrin molecules in a suitable biologically compatible aqueous buffer and placing it in a shallow dish or container or similar support; b) adding to a) a sufficient amount of hydrophilic polymers or derivatives thereof in a suitable biologically compatible aqueous buffer to create a solution having a hydrophilic polymer concentration of about 1 to about 20% (w/v) and a α-cyclodextrin concentration of about 0.1 to about 10% (w/v); c) mixing the solution of b) for a sufficient time to provide an inclusion step in which hydrophilic polymers or derivatives thereof and α-cyclodextrin molecules obtain a pseudopolyrotaxane configuration in which the hydrophilic polymers or derivatives thereof are included in the cavity of each of α-cyclodextrin molecule in a skewered manner; d) adding a photoinitiator to the solution of c) to create a final concentration of photoinitiator of between about 0.01 to about 0.1% (w/v); e) exposing the solution of d) to electromagnetic radiation at a wavelength specific to the photoinitiator for a sufficient amount of time to initiate the polymerization of the polymers in the solution; f) soaking the polymerized gel of e) for a sufficient period of time to remove any α-cyclodextrin which do not have the hydrophilic polymers or derivatives thereof are included in their cavities; and g) seeding a quantity of cells onto the polymerized gel off) at a density of between about 5000 to about 50,000 cells/cm² in a biologically compatible growth media.

In accordance with an embodiment, the present invention provides a method for making a 3-dimensional cell-encapsulated hydrogel comprising: a) obtaining a solution of α-cyclodextrin molecules in a suitable biologically compatible aqueous buffer and placing it in a container or similar support; b) adding to a) a sufficient amount of hydrophilic polymers or derivatives thereof in a suitable biologically compatible aqueous buffer to create a solution having a hydrophilic polymer concentration of about 1 to about 20% (w/v) and a α-cyclodextrin concentration of about 0.1 to about 10% (w/v); c) mixing the solution of b) for a sufficient time to provide an inclusion step in which hydrophilic polymers or derivatives thereof and α-cyclodextrin molecules obtain a pseudopolyrotaxane configuration in which the hydrophilic polymers or derivatives thereof are included in the cavity of each of α-cyclodextrin molecule in a skewered manner; d) adding a photoinitiator to the solution of c) to create a final concentration of photoinitiator of between about 0.01 to about 0.1% (w/v); e) seeding a quantity of cells into the solution of d) at a quantity of between about 500,000 to about 5×10⁶ cells in a biologically compatible growth media; and f) exposing the solution of e) to electromagnetic radiation at a wavelength specific to the photoinitiator for a sufficient amount of time to initiate the polymerization of the polymers in the solution.

In accordance with an embodiment, the present invention provides a method for making a multifunctional biomaterial comprising: a) obtaining a sufficient amount of hydrophobic biocompatible polymers or derivatives thereof in a suitable organic solvent to create a solution having a polymer concentration of about 0.1 to about 0.2 g/mL polymer and heating the solution to about 45 to 60° C.; b) adding to a) a solution of α-cyclodextrin molecules in a suitable polar aprotic solvent at a concentration of about 0.4 to 0.6 g/ml to create a mixture with a final concentration of α-cyclodextrin molecules in the mixture of between about 0.005 to about 0.008 g/ml; c) mixing the solution of b) for a sufficient time to provide an inclusion step in which the hydrophobic polymers or derivatives thereof and cyclodextrin molecules obtain a pseudopolyrotaxane configuration in which the polymers or derivatives thereof are included in the cavity of each of α-cyclodextrin molecule in a skewered manner; d) cooling the mixture of c) to room temperature; e) evaporating the organic solvent away from mixture of d) to produce a dried product; and f) washing the product of e) with water to remove excess α-cyclodextrin molecules.

In accordance with an embodiment, the present invention provides a multifunctional biomaterial comprising: one or more PCL polymers and one or more α-cyclodextrin molecules having a plurality of hydroxyl groups capable of being chemically substituted with another functional group or moiety selected from the group consisting of hydrophobic groups, hydrophilic groups, peptides, C₁-C₆ alkyl, C₂-C₆ alkenyl, C₂-C₆ alkynyl, C₁-C₆ hydroxyalkyl, C₁-C₆ alkoxy, C₁-C₆ alkoxy C₁-C₆ alkyl, C₁-C₆ alkylamino, di-C₁-C₆ alkylamino, C₁-C₆ dialkylamino C₁-C₆ alkyl, C₁-C₆ thioalkyl, C₂-C₆ thioalkenyl, C₂-C₆ thioalkynyl, C₆-C₂₂ aryloxy, C₂-C₆ acyloxy, C₂-C₆ thioacyl, C₁-C₆ amido, C₁-C₆ sulphonamido, C₁-C₆ carboxyl and derivatives, phosphonates and sulfones; wherein the one or more PCL polymers have at least 10 or more monomeric units; and wherein the one or more PCL polymers are included in the cavities of the one or more α-cyclodextrin molecules in a skewered manner to obtain a pseudopolyrotaxane configuration.

In accordance with a still further embodiment, the present invention provides a method for making a multifunctional nanofiber biomaterial comprising: a) obtaining a sufficient amount of hydrophobic biocompatible polymers or derivatives thereof in a suitable organic solvent to create a solution having a polymer concentration of about 0.1 to about 0.2 g/mL polymer and heating the solution to about 45° C. to 60° C.; b) adding to a) a solution of α-cyclodextrin molecules in a suitable polar aprotic solvent at a concentration of about 0.4 to 0.6 g/ml to create a mixture with a final concentration of α-cyclodextrin molecules in the mixture of between about 0.005 to about 0.008 g/ml; c) mixing the solution of b) for a sufficient time to provide an inclusion step in which the hydrophobic polymers or derivatives thereof and cyclodextrin molecules obtain a pseudopolyrotaxane configuration in which the polymers or derivatives thereof are included in the cavity of each of α-cyclodextrin molecule in a skewered manner; d) cooling the mixture of c) to room temperature; e) evaporating the organic solvent away from mixture of d) to produce a dried product; and f) washing the product of e) with water to remove excess α-cyclodextrin molecules; g) dissolving the product of e) in a mixture of dichloromethane and DMSO to create a solution having a concentration between about 5% to about 15% w/v of polymer product; and h) electrospinning the solution to create one or more nanofibers and allowing the fibers to dry.

### BRIEF DESCRIPTION OF THE DRAWINGS

FIG. 1. Synthesis and characterization of functionalized CDs. 1A, Dess-Martin periodinane (DMP) oxidizes α-CD to their aldehyde derivatives. Further oxidation by potassium peroxymonosulfate results in carboxylic acid derivatives. 1B, α-CDNH₂ is synthesized in two steps, first by activating α-CD with N,N′-carbonyldiimidazole, followed by its reaction with an excess of ethylenediamine 1C, 1H-NMR and MALDI-TOF spectra for α-CD, α-CD-CHO, α-CD-COOH, and α-CDNH₂.

FIG. 2. Biological activities and mechanical properties of PEGDA hydrogels with functionalized α-CDs. 2A, α-CDOH and its functional derivatives (COOH and NH₂) form inclusion complexes with poly(ethylene glycol) diacrylate (PEGDA). After threading, PEGDA is crosslinked to form a hydrogel. 2B, The live/dead staining of hMSCs encapsulated in hydrogels at different time intervals showing bioactivity of these gels. 2C, An array of hydrogels (PEGDA, 10% w/v) was synthesized with independently varied concentration of functional α-CDs (1% to 5%, w/v) at different pH. The compression moduli of the hydrogels did not significantly change at a specific pH by changing different functional α-CDs, except α-CDNH₂. At a higher pH, hydrogels with α-CDNH₂ produced softer gels, possibly due to a reaction of amine with the acrylate group. However, at an acidic pH, α-CDNH₂ produced a hydrogel with a similar stiffness value to that of other functional α-CDs.

FIG. 3. Biochemical analysis for chondrogenesis of hMSCs encapsulated in 3D hydrogels of PEGDA/α-CDs. Comparison of DNA content and cartilaginous ECM components in constructs with encapsulated hMSCs containing different amounts of α-CDs as indicated (0%, 1% and 5%, respectively) cultured in chondrogenic medium for 3 and 5 weeks. 3A, DNA content normalized by the dry weight of the respective constructs (μg/mg). GAG amount was quantified by DMMB assay and normalized to: 3B, DW (μg/mg), and 3C, DNA (μg/μg). Total collagen content was determined by hydroxyproline assay and normalized to: 3D, DW (μg/mg), and 3E, DNA (μg/μg). All data were presented as mean±standard deviation (n=3). Significantly higher (p≦0.5) values are shown with asterisk (*).

FIG. 4. Relative gene-expression values for chondrogenesis of hMSCs encapsulated in 3D hydrogels of PEGDA/α-CDs. PCR analysis showed the expression profile of chondrogenic gene markers for hMSCs in constructs including, 4A, Aggrecan, 4B, Collagen II, 4C, Sox9, and 4D, Collagen X. Significantly higher (p≦0.5) values are shown with asterisk (*).

FIG. 5. Structural characterization of functionalized β-CDs. 5A, ¹H-NMR and MALDI-TOF spectra for β-CDCOOH and β-CDCHO. 5B, ¹³C-NMR spectra for β-CDCOOH and α-CDCOOH.

FIG. 6. Swelling ratio of PEGDA hydrogels with functionalized α-CDs at various pH. An array of hydrogels (PEGDA, 10% w/v) was synthesized with independently varied concentration of functional α-CDs (1% to 5%, w/v) at pH 6.0, 7.4 and 9.0. The swelling ratio of the hydrogels did not significantly change at a specific pH by changing different functional α-CDs, except for α-CDNH₂.

FIG. 7. Application of functionalized α-CDs for creating cell-interactive molecular necklace, PEG hydrogels. 7A, Threading of α-CDNH₂ onto PEGDA chains followed by conjugation of a cell binding peptide, such as YRGDS. The cells can be either encapsulated in or cultured onto the surface of the hydrogel, which is synthesized by crosslinking PEGDA chains. 7B, The ninhydrin assay was performed on PEGDA/functionalized α-CD hydrogels to determine threading of α-CDs onto PEGDA chains. The ninhydrin assay produced a purple color in the presence of amine-containing hydrogels (shown as dark gray). 7C, FTIR-ATR spectra for different hydrogels (PEGDA, 5%, w/v and α-CDs, 10%, w/v) and their comparisons with control PEGDA polymer.

FIG. 8 is an illustration of the chemical structures of PCL and α-CD (8A), followed by inclusion complex (IC) formation (8B). The IC is electrospun into fibers (8C), and polystyrene nanobeads can be conjugated through the hydroxyl groups of α-CD on the fiber's surface (8D).

FIG. 9 depicts WAXD spectra (9A), FTIR-ATR spectra (9B) and ¹H-NMR spectra of α-CD, PCL and PCL-α-CD IC (9C).

FIG. 10. The hydroxyl groups of α-CD present in PCL-α-CD IC fibers can be conjugated with several biological or chemical moieties, including a fluorescent molecule. (10A) Step 1: Activation of α-CD with N,N′-carbonyldiimidazole (CDI) followed by its reaction with ethylenediamine. The hydroxyl groups are abundant and available for activation by N,N′-CDI in PCL-α-CD IC compared to only terminal hydroxyl groups of PCL. Step 2: Fluorescamine was conjugated to amine groups. (10B) Optical microscope images of electrospun fibers of PCL before (i) and after fluorescamine labeling (ii); PCL/α-CD fibers before (iii) and after fluorescamine labeling (iv).

FIG. 11 shows electrospun fibers of PCL-10% (w/v) in CH₂Cl₂/DMSO (17/9, v/v) with magnification X1 A), X10 B) & C), X20 D); PCL-α-CD IC-10% (w/v) in CH₂Cl₂/DMSO (2/3 v/v); with magnification X1 E), X10 F) & G), X20 H). These fibers were chemically modified with N,N′-carbonyldiimidazole in acetonitrile followed by conjugation of amine functionalized polystyrene nanobeads (200 nm diameter size). *denotes beads.

FIG. 12 is a series of graphs depicting the relative gene expression of some osteogenic markers during osteogenesis of hADSCs seeded on PCL and PCL-α-CD fibers, including RunX2 12A), osteopontin 12B), collagen type I 12C) and collagen type X 12D); biochemical assays showing DNA content 12E) and collagen deposition 12F) on the fibers.

### DETAILED DESCRIPTION OF THE INVENTION

In one or more embodiments the present invention provides synthetic biomaterial scaffolds that can be decorated with multiple chemical functionalities without altering the base hydrogel network. The inventive technology is useful for engineering tissue with many cell types, such as stem cells, and including, for example, human mesenchymal stem cells (hMSCs).

In accordance with one or more embodiments, the present inventors have designed a multifunctional biomaterial comprising electrospun nanofibers based on the inclusion complex of PCL-α-cyclodextrin (PCL-α-CD) in a pseudopolyrotaxane conformation, providing both structural support and multiple functionalities for further conjugation of bioactive components. This inventive strategy is independent of any chemical modification of the PCL main chain, and electrospinning of PCL-α-CD is as easy as electrospinning PCL. Included herein is a description of the synthesis of the PCL-α-CD biomaterials, the elucidated composition and structure, and a demonstration of the utility of functional groups on the nanofibers biomaterial by conjugating a fluorescent small molecule and a polymeric-nanobead to the nanofibers of the present invention.

Furthermore, in one or more embodiments, the application of PCL-α-CD nanofibers biomaterials of the present invention are shown to be suitable for a variety of biological applications, including, for example, promoting osteogenic differentiation of human adipose-derived stem cells (hADSCs), which induced a higher level of expression of osteogenic markers and enhanced production of extracellular matrix (ECM) proteins or molecules compared to control PCL fibers.

As disclosed herein, in one or more embodiments, amine- and carboxylic acid-functionalized α-CD molecules from the commercially available alcohol substituted α-CD, were synthesized and utilized to create an array of PEG/α-CD functionalized hydrogel biomaterials of the present invention. These inventive hydrogel biomaterials supported cartilage tissue formation at the lower concentrations of functionalized α-CDs, regardless of the type of functionalities. By increasing the concentration of functionalized group, the hydroxyl groups-substituted PEG/α-CD hydrogels enhanced cartilage tissue formation, while the carboxylic acid-substituted PEG/α-CD hydrogels suppressed the productions of glycosaminoglycans (GAGs) and collagen.

In alternative embodiments, chemical functional groups may be chosen for the desired cell response, tissue development or scaffold properties.

In accordance with an embodiment, the present invention provides a multifunctional biomaterial comprising: one or more biocompatible polymers and one or more α-cyclodextrin molecules having a plurality of hydroxyl groups capable of being chemically substituted with another functional group or moiety; wherein the one or more biocompatible polymers have at least 10 or more monomeric units; and wherein the one or more biocompatible polymers are included in the cavities (i.e., an inclusion complex(s) (IC)) of the one or more α-cyclodextrin molecules in a skewered manner to obtain a pseudopolyrotaxane configuration.

The multifunctional aspects of one or more embodiments of the present invention are due to the ability to substitute the hydroxyl groups of the α-CD molecules with another functional group or moiety, thus changing the physical and chemical characteristics of the material without necessarily altering the chemical structure of the backbone polymer. The functional groups can be substituted with any suitable compound or moiety, including, for example, hydrophobic groups, hydrophilic groups, peptides, C₁-C₆ alkyl, C₂-C₆ alkenyl, C₂-C₆ alkynyl, C₁-C₆ hydroxyalkyl, C₁-C₆ alkoxy, C₁-C₆ alkoxy C₁-C₆ alkyl, C₁-C₆ alkylamino, di-C₁-C₆ alkylamino, C₁-C₆ dialkylamino C₁-C₆ alkyl, C₁-C₆ thioalkyl, C₂-C₆ thioalkenyl, C₂-C₆ thioalkynyl, C₆-C₂₂ aryloxy, C₂-C₆ acyloxy, C₂-C₆ thioacyl, C₁-C₆ amido, C₁-C₆ sulphonamido, C₁-C₆ carboxyl and derivatives, phosphonates and sulfones.

The biocompatible polymers used in the multifunctional biomaterials can be hydrophilic and hydrophobic. Examples of biocompatible polymers useful in the biomaterials of the present invention include, Poly(ethylene glycol), Poly(propylene glycol), Poly(methyl vinyl ether), Oligoethylene, Poly(isobutylene) Poly(tetrahydrofuran) Poly(oxytrimethylene), Poly(dimethylsiloxsane), Poly(dimethylsilane), Nylon 6, Nylon 11, Poly(acrylonitrile), Squalane, Poly(1,3-dioxolane), Poly(iminooligomethylene), Poly(1-lysine), Polyethyleneimine, Poly(adipate), Poly(l-caprolactone), Poly(L-lactic acid), or derivatives thereof.

Therefore, in accordance with an embodiment, the present invention provides a multifunctional biomaterial comprising: one or more polycaprolactone (PCL) polymers and one or more α-cyclodextrin molecules having a plurality of hydroxyl groups capable of being chemically substituted with another functional group or moiety selected from the group consisting of hydrophobic groups, hydrophilic groups, peptides, C₁-C₆ alkyl, C₂-C₆ alkenyl, C₂-C₆ alkynyl, C₁-C₆ hydroxyalkyl, C₁-C₆ alkoxy, C₁-C₆ alkoxy C₁-C₆ alkyl, C₁-C₆ alkylamino, di-C₁-C₆ alkylamino, C₁-C₆ dialkylamino C₁-C₆ alkyl, C₁-C₆ thioalkyl, C₂-C₆ thioalkenyl, C₂-C₆ thioalkynyl, C₆-C₂₂ aryloxy, C₂-C₆ acyloxy, C₂-C₆ thioacyl, C₁-C₆ amido, C₁-C₆ sulphonamido, C₁-C₆ carboxyl and derivatives, phosphonates and sulfones; wherein the one or more PCL polymers have at least 10 or more monomeric units; and wherein the one or more PCL polymers are included in the cavities of the one or more α-cyclodextrin molecules in a skewered manner to obtain a pseudopolyrotaxane configuration.

In accordance with another embodiment, the present invention provides a hydrogel system comprising one or more poly(ethylene) glycol polymers and/or derivatives thereof and one or more α-cyclodextrin molecules having a plurality of hydroxyl groups capable of being chemically substituted with another functional group or moiety selected from the group consisting of hydrophobic groups, hydrophilic groups, peptides, C₁-C₆ alkyl, C₂-C₆ alkenyl, C₂-C₆ alkynyl, C₁-C₆ hydroxyalkyl, C₁-C₆ alkoxy, C₁-C₆ alkoxy C₁-C₆ alkyl, C₁-C₆ alkylamino, di-C₁-C₆ alkylamino, C₁-C₆ dialkylamino C₁-C₆ alkyl, C₁-C₆ thioalkyl, C₂-C₆ thioalkenyl, C₂-C₆ thioalkynyl, C₆-C₂₂ aryloxy, C₂-C₆ acyloxy, C₂-C₆ thioacyl, C₁-C₆ amido, C₁-C₆ sulphonamido, C₁-C₆ carboxyl and derivatives, phosphonates and sulfones; wherein the one or more poly(ethylene glycol) polymers have at least 10 or more monomeric units; and wherein the one or more poly(ethylene glycol) polymers are included in the cavities of the one or more α-cyclodextrin molecules in a skewered manner to obtain a pseudopolyrotaxane configuration.

As used herein, examples of the term “alkyl” preferably include a C₁₋₆ alkyl (e.g., methyl, ethyl, propyl, isopropyl, butyl, isobutyl, sec-butyl, tert-butyl, pentyl, hexyl, etc.) and the like.

As used herein, examples of the term “alkenyl” preferably include C₂₋₆ alkenyl (e.g., vinyl, allyl, isopropenyl, 1-butenyl, 2-butenyl, 3-butenyl, 2-methyl-2-propenyl, 1-methyl-2-propenyl, 2-methyl-1-propenyl, etc.) and the like.

As used herein, examples of the term “alkynyl” preferably include C₂₋₆ alkynyl (e.g., ethynyl, propargyl, 1-butynyl, 2-butynyl, 3-butynyl, 1-hexynyl, etc.) and the like.

Examples of the term “cycloalkyl” preferably include a C₃₋₈ cycloalkyl (e.g., a cyclopropyl, cyclobutyl, cyclopentyl, cyclohexyl, etc.) and the like.

Examples of the term “aryl” preferably include a C₆₋₁₄ aryl (e.g., a phenyl, 1-naphthyl, a 2-naphthyl, 2-biphenylyl group, 3-biphenylyl, 4-biphenylyl, 2-anthracenyl, etc.) and the like.

Examples of the term “arylalkyl” preferably include a C₆₋₁₄ arylalkyl (e.g., benzyl, phenylethyl, diphenylmethyl, 1-naphthylmethyl, 2-naphthylmethyl, 2,2-diphenylethyl, 3-phenylpropyl, 4-phenylbutyl, 5-phenylpentyl, etc.) and the like.

The term “hydroxyalkyl” embraces linear or branched alkyl groups having one to about ten carbon atoms any one of which may be substituted with one or more hydroxyl groups.

The term “alkylamino” includes monoalkylamino. The term “monoalkylamino” means an amino, which is substituted with an alkyl as defined herein. Examples of monoalkylamino substituents include, but are not limited to, methylamino, ethylamino, isopropylamino, t-butylamino, and the like. The term “dialkylamino” means an amino, which is substituted with two alkyls as defined herein, which alkyls can be the same or different. Examples of dialkylamino substituents include dimethylamino, diethylamino, ethylisopropylamino, diisopropylamino, dibutylamino, and the like.

The terms “alkylthio,” “alkenylthio” and “alkynylthio” mean a group consisting of a sulphur atom bonded to an alkyl-, alkenyl- or alkynyl-group, which is bonded via the sulphur atom to the entity to which the group is bonded.

A “rotaxane” is a mechanically-interlocked molecular architecture consisting of a “dumbbell shaped molecule” which is threaded through a macrocyclic molecule. The name is derived from the Latin for wheel (rota) and axle (axis). As used herein, the term “pseudopolyrotaxane” means an interlocked set of molecules where the PEG polymer “thread” is threaded through the cavity of the α-CD molecule (the macrocycle), however, the inventive structure lacks the “dumbell ends” as ordinarily understood, hence the use of the prefix “-pseudo.” In addition, the use of the prefix “poly” is intended to convey the concept that the hydrogel system can comprise any number of PEG “threads” having one or more α-CD molecules “threaded” or “skewered” onto them. Further, in accordance with one or more embodiments, these pseudopolyrotaxane polymer molecules can be cross-linked to each other to form a network.

By “hydrogel” is meant a water-swellable polymeric matrix that can absorb water to form elastic gels, wherein “matrices” are three-dimensional networks of macromolecules held together by covalent or noncovalent crosslinks. On placement in an aqueous environment, dry hydrogels swell by the acquisition of liquid therein to the extent allowed by the degree of cross-linking.

As used herein, the terms “stability” and “stable” in the context of a liquid formulation comprising a biopolymer of interest that is resistant to thermal and chemical aggregation, degradation or fragmentation under given manufacture, preparation, transportation and storage conditions, such as, for one month, for two months, for three months, for four months, for five months, for six months or more. The “stable” formulations of the invention retain biological activity equal to or more than 80%, 85%, 90%, 95%, 98%, 99% or 99.5% under given manufacture, preparation, transportation and storage conditions. The stability of said preparation can be assessed by degrees of aggregation, degradation or fragmentation by methods known to those skilled in the art.

A biologically compatible polymer refers to a polymer which is functionalized to serve as a composition for creating an implant. The polymer is one that is a naturally occurring polymer or one that is not toxic to the host. The polymer can, e.g., contain at least an imide. The polymer may be a homopolymer where all monomers are the same or a hetereopolymer containing two or more kinds of monomers. The terms “biocompatible polymer,” “biocompatible cross-linked polymer matrix” and “biocompatibility” when used in relation to the instant polymers are art-recognized are considered equivalent to one another, including to biologically compatible polymer. For example, biocompatible polymers include polymers that are neither toxic to the host (e.g., an animal or human), nor degrade (if the polymer degrades at a rate that produces monomeric or oligomeric subunits or other byproducts at toxic concentrations in the host).

“Polymer” is used to refer to molecules composed of repeating monomer units, including homopolymers, block copolymers, heteropolymers, random copolymers, graft copolymers and so on. “Polymers” also include linear polymers as well as branched polymers, with branched polymers including highly branched, dendritic, and star polymers.

A monomer is the basic repeating unit in a polymer. A monomer may itself be a monomer or may be dimer or oligomer of at least two different monomers, and each dimer or oligomer is repeated in a polymer.

A “polymerizing initiator” refers to any substance that can initiate polymerization of monomers or macromers by, for example, free radical generation. The polymerizing initiator often is an oxidizing agent. Exemplary polymerization initiators include those which are activated by exposure to, for example, electromagnetic radiation or heat. Polymerization initiators can also be used and are described, e.g., in U.S. Patent Application Publication No. 2010/0137241, which is incorporated by reference in entirety.

This disclosure is directed, at least in part, to polymers, matrices, and gels, and methods of making and using matrices, polymers and gels. Gels, networks, scaffolds, films and the like of interest made with the composition(s) of interest encourage cell, tissue and organ integration and growth. The optional presence of cells, such as stem cells, enhances cell, tissue, and organ integration and growth.

In accordance with an embodiment, the present invention provides a hydrogel system as described above, wherein the one or more poly(ethylene glycol) polymers are block copolymers.

In accordance with another embodiment, the present invention provides a hydrogel system as described above, wherein the one or more poly(ethylene glycol) polymers are mono, or disubstituted with one or more acrylate groups.

Significant to the hydrogel system of the present invention is the enhanced integration with the surrounding tissue to increase stability and bonding to a biological surface and to formation of new tissue.

The instant invention provides for ex vivo polymerization techniques to form scaffolds and so on that can be molded to take the desired shape of a tissue defect, promote tissue development by stimulating native cell repair, and can be potentially implanted by minimally invasive injection.

An “active agent” and a “biologically active agent” are used interchangeably herein to refer to a chemical or biological compound that induces a desired pharmacological and/or physiological effect, wherein the effect may be prophylactic or therapeutic. The terms also encompass pharmaceutically acceptable, pharmacologically active derivatives of those active agents specifically mentioned herein, including, but not limited to, salts, esters, amides, prodrugs, active metabolites, analogs and the like. When the terms “active agent,” “pharmacologically active agent” and “drug” are used, then, it is to be understood that the invention includes the active agent per se as well as pharmaceutically acceptable, pharmacologically active salts, esters, amides, prodrugs, metabolites, analogs etc. The active agent can be a biological entity, such as a virus or cell, whether naturally occurring or manipulated, such as transformed.

“Biocompatible polymer,” biocompatible cross-linked polymer matrix and biocompatibility are art-recognized terms. For example, biocompatible polymers include polymers that are neither themselves toxic to the host (e.g., and animal or human), nor degrade (if the polymer degrades) at a rate that produces monomeric or oligomeric subunits or other byproducts at toxic concentrations in the host. In certain embodiments of the present invention, biodegradation generally involves degradation of the polymer in an organism, e.g., into its monomeric subunits, which may be known to be effectively non-toxic. Intermediate oligomeric products resulting from such degradation may have different toxicological properties, however, or biodegradation may involve oxidation or other biochemical reactions that generate molecules other than monomeric subunits of the polymer. Consequently, in certain embodiments, toxicology of a biodegradable polymer intended for in vivo use, such as implantation or injection into a patient, may be determined after one or more toxicity analyses. It is not necessary that any subject composition have a purity of 100% to be deemed biocompatible; indeed, it is only necessary that the subject compositions are biocompatible as set forth above. Hence, a subject composition may comprise polymers comprising 99%, 98%, 97%, 96%, 95%, 90%, 85%, 80%, 75% or even less of biocompatible polymers, e.g., including polymers and other materials and excipients described herein, and still be biocompatible.

“Biodegradable” is art-recognized, and includes monomers, polymers, polymer matrices, gels, compositions and formulations, such as those described herein, that are intended to degrade during use, such as in vivo. Biodegradable polymers and matrices typically differ from non-biodegradable polymers in that the former may be degraded during use. In certain embodiments, such use involves in vivo use, such as in vivo therapy, and in other certain embodiments, such use involves in vitro use. In general, degradation attributable to biodegradability involves the degradation of a biodegradable polymer into its component subunits, or digestion, e.g., by a biochemical process, of the polymer into smaller, non-polymeric subunits. In certain embodiments, two different types of biodegradation may generally be identified. For example, one type of biodegradation may involve cleavage of bonds (whether covalent or otherwise) in the polymer backbone. In such biodegradation, monomers and oligomers typically result, and even more typically, such biodegradation occurs by cleavage of a bond connecting one or more of subunits of a polymer. In contrast, another type of biodegradation may involve cleavage of a bond (whether covalent or otherwise) internal to a side chain or that connects a side chain, functional group and so on to the polymer backbone. For example, a therapeutic agent, biologically active agent, or other chemical moiety attached as a side chain to the polymer backbone may be released by biodegradation. In certain embodiments, one or the other or both general types of biodegradation may occur during use of a polymer. As used herein, the term “biodegradation” encompasses both general types of biodegradation.

The degradation rate of a biodegradable polymer often depends in part on a variety of factors, including the chemical identity of the linkage responsible for any degradation, the molecular weight, crystallinity, biostability, and degree of cross-linking of such polymer, the physical characteristics of the implant, shape and size, and the mode and location of administration. For example, the greater the molecular weight, the higher the degree of crystallinity, and/or the greater the biostability, the biodegradation of any biodegradable polymer is usually slower. The term “biodegradable” is intended to cover materials and processes also termed “bioerodible.”

In certain embodiments, polymeric formulations of the present invention biodegrade within a period that is acceptable in the desired application. In certain embodiments, such as in vivo therapy, such degradation occurs in a period usually less than about five years, one year, six months, three months, one month, fifteen days, five days, three days, or even one day on exposure to a physiological solution with a pH between 6 and 8 having a temperature of between about 25° C. to 37° C. In other embodiments, the polymer degrades in a period of between about one hour and several weeks, depending on the desired application. In some embodiments, the polymer or polymer matrix may include a detectable agent that is released on degradation.

Cross-linked herein refers to a composition containing intermolecular cross-links and optionally intramolecular cross-links, arising from, generally, the formation of covalent bonds. Covalent bonding between two cross-linkable components may be direct, in which case an atom in one component is directly bound to an atom in the other component, or it may be indirect, through a linking group. A cross-linked gel or polymer matrix may, in addition to covalent, also include intermolecular and/or intramolecular noncovalent bonds such as hydrogen bonds and electrostatic (ionic) bonds.

“Functionalized” refers to a modification of an existing molecular segment or group to generate or to introduce a new reactive or more reactive group (e.g., imide group) that is capable of undergoing reaction with another functional group (e.g., an amine group) to form a covalent bond. For example, carboxylic acid groups can be functionalized by reaction with a carbodiimide and an imide reagent using known procedures to provide a new reactive functional group in the form of an imide group substituting for the hydrogen in the hydroxyl group of the carboxyl function.

“Gel” refers to a state of matter between liquid and solid, and is generally defined as a cross-linked polymer network swollen in a liquid medium. Typically, a gel is a two-phase colloidal dispersion containing both solid and liquid, wherein the amount of solid is greater than that in the two-phase colloidal dispersion referred to as a “sol.” As such, a “gel” has some of the properties of a liquid (i.e., the shape is resilient and deformable) and some of the properties of a solid (i.e., the shape is discrete enough to maintain three dimensions on a two-dimensional surface).

Hydrogels consist of hydrophilic polymers cross-linked to from a water-swollen, insoluble polymer network. Cross-linking can be initiated by many physical or chemical mechanisms. Photopolymerization is a method of covalently crosslink polymer chains, whereby a photoinitiator and polymer solution (termed “pre-gel” solution) are exposed to a light source specific to the photoinitiator. On activation, the photoinitiator reacts with specific functional groups in the polymer chains, crosslinking them to form the hydrogel. The reaction is rapid (3-5 minutes) and proceeds at room and body temperature. Photoinduced gelation enables spatial and temporal control of scaffold formation, permitting shape manipulation after injection and during gelation in vivo. Cells and bioactive factors can be easily incorporated into the hydrogel scaffold by simply mixing with the polymer solution prior to photogelation.

Hydrogels of interest can be semi-interpenetrating networks that promote cell, tissue and organ repair while discouraging scar formation. The hydrogels of interest also are configured to have a viscosity that will enable the gelled hydrogel to remain affixed on or in the cell, tissue or organ, or surface. Viscosity can be controlled by the monomers and polymers used, by the level of water trapped in the hydrogel, and by incorporated thickeners, such as biopolymers, such as proteins, lipids, saccharides and the like. An example of such a thickener is hyaluronic acid or collagen.

“Incorporated,” “encapsulated,” and “entrapped” are art-recognized when used in reference to a therapeutic agent, dye, or other material and a polymeric composition, such as a composition of the present invention. In certain embodiments, these terms include incorporating, formulating or otherwise including such agent into a composition that allows for sustained release of such agent in the desired application. The terms may contemplate any manner by which a therapeutic agent or other material is incorporated into a polymer matrix, including, for example, attached to a monomer of such polymer (by covalent or other binding interaction) and having such monomer be part of the polymerization to give a polymeric formulation, distributed throughout the polymeric matrix, appended to the surface of the polymeric matrix (by covalent or other binding interactions), encapsulated inside the polymeric matrix, etc. The term “co-incorporation” or “co-encapsulation” refers to the incorporation of a therapeutic agent or other material and at least one other therapeutic agent or other material in a subject composition.

It will be understood that “substitution” or “substituted with” includes the implicit proviso that such substitution is in accordance with the permitted valency of the substituted atom and the substituent, and that the substitution results in a stable compound, e.g., which does not spontaneously undergo transformation, such as by rearrangement, cyclization, elimination, or other reaction.

The term “substituted” is also contemplated to include all permissible substituents of organic compounds such as the imide reagent of interest. In a broad aspect, the permissible substituents include acyclic and cyclic, branched and unbranched, carbocyclic and heterocyclic, aromatic and nonaromatic substituents of organic compounds. Illustrative substituents include, for example, those described herein. The permissible substituents may be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

In accordance with an embodiment, the present invention provides a hydrogel system as described above, wherein the one or more α-cyclodextrin molecules have their hydroxyl groups substituted with an aldehyde, a carboxylic acid group, or an amino group.

A functional group or a moiety which can be used for substitution is one capable of mediating formation of a polymer or reaction with a surface or other molecule. Functional groups include the various radicals and chemical entities taught herein, and include alkenyl moieties such as acrylates, methacrylates, dimethacrylates, oligoacrylates, oligomethacrylates, ethacrylates, itaconates or acrylamides. Further functional groups include aldehydes. Other functional groups may include ethylenically unsaturated monomers including, for example, alkyl esters of acrylic or methacrylic acid such as methyl methacrylate, ethyl methacrylate, butyl methacrylate, ethyl acrylate, butyl acrylate, hexyl acrylate, n-octyl acrylate, lauryl methacrylate, 2-ethylhexyl methacrylate, nonyl acrylate, benzyl methacrylate, the hydroxyalkyl esters of the same acids such as 2-hydroxyethyl acrylate, 2-hydroxyethyl methacrylate, and 2-hydroxypropyl methacrylate, the nitrile and amides of the same acids such as acrylonitrile, methacrylonitrile, and methacrylamide, vinyl acetate, vinyl propionate, vinylidene chloride, vinyl chloride, and vinyl aromatic compounds such as styrene, t-butyl styrene and vinyl toluene, dialkyl maleates, dialkyl itaconates, dialkyl methylene-malonates, isoprene, and butadiene. Suitable ethylenically unsaturated monomers containing carboxylic acid groups include acrylic monomers such as acrylic acid, methacrylic acid, ethacrylic acid, itaconic acid, maleic acid, fumaric acid, monoalkyl itaconate including monomethyl itaconate, monoethyl itaconate, and monobutyl itaconate, monoalkyl maleate including monomethyl maleate, monoethyl maleate, and monobutyl maleate, citraconic acid, and styrene carboxylic acid. Suitable polyethylenically unsaturated monomers include butadiene, isoprene, allylmethacrylate, diacrylates of alkyl diols such as butanediol diacrylate and hexanediol diacrylate, divinyl benzene, and the like.

In accordance with an embodiment, the present invention provides a hydrogel system described above, wherein the one or more poly(ethylene glycol) polymers is poly(ethylene glycol) diacrylate (PEGDA).

In some embodiments, a monomeric unit of a biologically compatible polymer may be functionalized through one or more thio, carboxylic acid or alcohol moieties located on a monomer of the biopolymer.

Cross-linked polymer matrices of the present invention may include and form hydrogels. The water content of a hydrogel may provide information on the pore structure. Further, the water content may be a factor that influences, for example, the survival of encapsulated cells within the hydrogel. The amount of water that a hydrogel is able to absorb may be related to the cross-linking density and/or pore size. In accordance with an embodiment, the present invention provides a hydrogel system as described above, wherein the hydrogel is cross-linked. The polymer chains can be crosslinked via the terminal ends of the polymers and not through the α-cyclodextrin molecules.

The compositions of the present invention may comprise monomers, macromers, oligomers, polymers, or a mixture thereof. The polymer compositions can consist solely of covalently crosslinkable polymers, or ionically crosslinkable polymers, or polymers crosslinkable by redox chemistry, or polymers crosslinked by hydrogen bonding, or any combination thereof. The reagents should be substantially hydrophilic and biocompatible.

In some embodiments, the number of each of the functional groups per polymeric unit may be at least one moiety per about 10 monomeric units, at least about 2 moieties per about 10 monomeric units up through one or more functional groups per monomer. Alternatively, the number of functional groups per polymeric unit may be at least one moiety per about 12 monomeric units, per about 14 monomeric units or more.

Cytotoxicity of the biomaterials of the present invention may be evaluated with any suitable cells, such as fibroblasts, by, for example, using a live-dead fluorescent cell assay and MTT, a compound that actively metabolizing cells convert from yellow to purple, as taught hereinabove, and as known in the art.

In one aspect of this invention, a composition comprising a multifunctional biomaterial and one or more biologically active agents may be prepared. The biologically active agent may vary widely with the intended purpose for the composition. The term active is art-recognized and refers to any moiety that is a biologically, physiologically, or pharmacologically active substance that acts locally or systemically in a subject. Examples of biologically active agents, that may be referred to as “drugs”, are described in well-known literature references such as the Merck Index, the Physicians' Desk Reference, and The Pharmacological Basis of Therapeutics, and they include, without limitation, medicaments; vitamins; mineral supplements; substances used for the treatment, prevention, diagnosis, cure or mitigation of a disease or illness; substances which affect the structure or function of the body; or pro-drugs, which become biologically active or more active after they have been placed in a physiological environment. Various forms of a biologically active agent may be used which are capable of being released the subject composition, for example, into adjacent tissues or fluids upon administration to a subject. In some embodiments, a biologically active agent may be used in cross-linked polymer matrix of this invention, to, for example, promote cartilage formation. In other embodiments, a biologically active agent may be used in cross-linked polymer matrix of this invention, to treat, ameliorate, inhibit, or prevent a disease or symptom, in conjunction with, for example, promoting cartilage formation.

Further examples of biologically active agents include, without limitation, enzymes, receptor antagonists or agonists, hormones, growth factors, autogenous bone marrow, antibiotics, antimicrobial agents, and antibodies. The term “biologically active agent” is also intended to encompass various cell types and genes that can be incorporated into the compositions of the invention.

In certain embodiments, the subject compositions comprise about 1% to about 75% or more by weight of the total composition, alternatively about 2.5%, 5%, 10%, 20%, 30%, 40%, 50%, 60% or 70%, of a biologically active agent.

Non-limiting examples of biologically active agents include following: adrenergic blocking agents, anabolic agents, androgenic steroids, antacids, anti-asthmatic agents, anti-allergenic materials, anti-cholesterolemic and anti-lipid agents, anti-cholinergics and sympathomimetics, anti-coagulants, anti-convulsants, anti-diarrheal, anti-emetics, anti-hypertensive agents, anti-infective agents, anti-inflammatory agents such as steroids, non-steroidal anti-inflammatory agents, anti-malarials, anti-manic agents, anti-nauseants, anti-neoplastic agents, anti-obesity agents, anti-parkinsonian agents, anti-pyretic and analgesic agents, anti-spasmodic agents, anti-thrombotic agents, anti-uricemic agents, anti-anginal agents, antihistamines, anti-tussives, appetite suppressants, benzophenanthridine alkaloids, biologicals, cardioactive agents, cerebral dilators, coronary dilators, decongestants, diuretics, diagnostic agents, erythropoietic agents, estrogens, expectorants, gastrointestinal sedatives, agents, hyperglycemic agents, hypnotics, hypoglycemic agents, ion exchange resins, laxatives, mineral supplements, mitotics, mucolytic agents, growth factors, neuromuscular drugs, nutritional substances, peripheral vasodilators, progestational agents, prostaglandins, psychic energizers, psychotropics, sedatives, stimulants, thyroid and anti-thyroid agents, tranquilizers, uterine relaxants, vitamins, antigenic materials, and prodrugs.

Further, recombinant or cell-derived proteins may be used, such as recombinant beta-glucan; bovine immunoglobulin concentrate; bovine superoxide dismutase; formulation comprising fluorouracil, epinephrine, and bovine collagen; recombinant hirudin (r-Hir), HIV-1 immunogen; recombinant human growth hormone recombinant EPO (r-EPO); gene-activated EPO (GA-EPO); recombinant human hemoglobin (r-Hb); recombinant human mecasermin (r-IGF-1); recombinant interferon α; lenograstim (G-CSF); olanzapine; recombinant thyroid stimulating hormone (r-TSH); and topotecan.

Still further, the following listing of peptides, proteins, and other large molecules may also be used, such as interleukins 1 through 18, including mutants and analogues; interferons a, y, and which may be useful for cartilage regeneration, hormone releasing hormone (LHRH) and analogues, gonadotropin releasing hormone transforming growth factor (TGF); fibroblast growth factor (FGF); tumor necrosis factor-α); nerve growth factor (NGF); growth hormone releasing factor (GHRF), epidermal growth factor (EGF), connective tissue activated osteogenic factors, fibroblast growth factor homologous factor (FGFHF); hepatocyte growth factor (HGF); insulin growth factor (IGF); invasion inhibiting factor-2 (IIF-2); bone morphogenetic proteins 1-7 (BMP 1-7); somatostatin; thymosin-a-y-globulin; superoxide dismutase (SOD); and complement factors, and biologically active analogs, fragments, and derivatives of such factors, for example, growth factors.

Members of the transforming growth factor (TGF) supergene family, which are multifunctional regulatory proteins, may be incorporated in a polymer matrix of the present invention. Members of the TGF supergene family include the beta transforming growth factors (for example, TGF-131, TGF-132, TGF-133); bone morphogenetic proteins (for example, BMP-1, BMP-2, BMP-3, BMP-4, BMP-5, BMP-6, BMP-7, BMP-8, BMP-9); heparin-binding growth factors (for example, fibroblast growth factor (FGF), epidermal growth factor (EGF), platelet-derived growth factor (PDGF), insulin-like growth factor (IGF)), (for example, Inhibin A, Inhibin B), growth differentiating factors (for example, GDF-1); and Activins (for example, Activin A, Activin B, Activin AB). Growth factors can be isolated from native or natural sources, such as from mammalian cells, or can be prepared synthetically, such as by recombinant DNA techniques or by various chemical processes. In addition, analogs, fragments, or derivatives of these factors can be used, provided that they exhibit at least some of the biological activity of the native molecule. For example, analogs can be prepared by expression of genes altered by site-specific mutagenesis or other genetic engineering techniques.

In accordance with an embodiment, the present invention provides a hydrogel biomaterial as described above, wherein the one or more α-cyclodextrin molecules have their hydroxyl groups substituted with an integrin binding peptide.

In accordance with another embodiment, the present invention provides a hydrogel system as described above, wherein the integrin binding peptide is YRGDS (SEQ ID NO: 17).

Various forms of the biologically active agents may be used. These include, without limitation, such forms as uncharged molecules, molecular complexes, salts, ethers, esters, amides, prodrug forms and the like, which are biologically activated when implanted, injected or otherwise placed into a subject.

In certain embodiments, other materials may be incorporated into subject compositions in addition to one or more biologically active agents. For example, plasticizers and stabilizing agents known in the art may be incorporated in compositions of the present invention. In certain embodiments, additives such as plasticizers and stabilizing agents are selected for their biocompatibility or for the resulting physical properties of the reagents, the setting or gelling matrix or the set or gelled matrix.

The multifunctional biomaterial compositions will be formulated, dosed and administered in a manner consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “therapeutically effective amount” of the biopolymer to be administered will be governed by such considerations, and can be the minimum amount necessary to prevent, ameliorate or treat a disorder of interest. As used herein, the term “effective amount” is an equivalent phrase refers to the amount of a therapy (e.g., a prophylactic or therapeutic agent), which is sufficient to reduce the severity and/or duration of a disease, ameliorate one or more symptoms thereof, prevent the advancement of a disease or cause regression of a disease, or which is sufficient to result in the prevention of the development, recurrence, onset, or progression of a disease or one or more symptoms thereof, or enhance or improve the prophylactic and/or therapeutic effect(s) of another therapy (e.g., another therapeutic agent) useful for treating a disease. For example, a treatment using the hydrogels of the present invention can increase the use of a joint in a host, based on baseline of the injured or diseases joint, by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%. In another embodiment, an effective amount of a therapeutic or a prophylactic hydrogel of the present invention reduces the symptoms of a disease, such as a symptom of arthritis by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%. Also used herein as an equivalent is the term, “therapeutically effective amount.”

Biologically active agents and other additives may be incorporated into α-CD that have the polymers included through them via substitution of the hydroxyl groups of the α-CD molecules in the hydrogel composition.

The hydrogel biomaterial compositions of the present invention can also be used to deliver various types of living cells (e.g., a mesenchymal stem cell, a cardiac stem cell, a liver stem cell, a retinal stem cell, and an epidermal stem cell) or genes to a desired site of administration to form new tissue. The term “genes” as used herein is intended to encompass genetic material from natural sources, synthetic nucleic acids, DNA, antisense DNA and RNA.

For example, mesenchymal stem cells, such as hMSCs, can be delivered using polymer matrices made using the hydrogel system described herein, to produce cells of the same type as the tissue into which they are delivered. MSCs may not be differentiated and therefore may differentiate to form various types of new cells due to the presence of an active agent or the effects (chemical, physical, etc.) of the local tissue environment. Examples of MSCs include osteoblasts, chondrocytes, and fibroblasts. For example, osteoblasts can be delivered to the site of a bone defect to produce new bone; chondrocytes can be delivered to the site of a cartilage defect to produce new cartilage; fibroblasts can be delivered to produce collagen wherever new connective tissue is needed; neurectodermal cells can be delivered to form new nerve tissue; epithelial cells can be delivered to form new epithelial tissues, such as liver, pancreas etc.

The cells may be either allogeneic or xenogeneic in origin. The compositions can be used to deliver cells of species that are genetically modified.

In some embodiments, the compositions of the invention may not easily be degraded in vivo. Thus, cells entrapped within the hydrogel compositions will be isolated from the host cells and, as such, will not provoke or will delay an immune response in the host.

To entrap the cells or genes within the inventive hydrogels, the cells or genes may, for example, be premixed with a reagent composition or optionally with a mixture prior to forming a cross-linked polymer matrix, thereby entrapping the cells or genes within the matrix.

In accordance with an embodiment, the present invention provides a hydrogel system as described above, wherein the hydrogel is 2-dimensional.

In some embodiments, compositions disclosed herein may be positioned in a surgically created defect that is to be reconstructed, and is to be left in that position after the reconstruction has been completed. The present invention may be suitable for use with local tissue reconstructions.

In certain embodiments, the inventive hydrogels can be formed into desired structures, such as films, foams, scaffolds or other three-dimensional structures of interest. In such circumstances, other materials may be incorporated into subject compositions, in addition to one or more biologically active agents.

In accordance with an embodiment, the present invention provides a hydrogel system as described above, wherein the hydrogel is 3-dimensional.

The multifunctional biomaterial compositions disclosed herein may be used in any number of tissue repair applications. The hydrogels of the invention can also be used for augmentation of soft or hard tissue within the body of a mammalian subject.

In one embodiment, the repair of damaged tissue may be carried out within the context of any standard surgical process allowing access to and repair of the tissue, including open surgery and laparoscopic techniques. Once the damaged tissue is accessed, a hydrogel composition of the invention is placed in contact with the damaged tissue along with any surgically acceptable patch or implant, if needed.

In accordance with an embodiment, the present invention provides a method for making a hydrogel biomaterial comprising: a) obtaining a saturated solution of α-cyclodextrin molecules in a suitable biologically compatible aqueous buffer; b) adding to a) a sufficient amount of poly(ethylene glycol) (PEG) polymers or derivatives thereof in a suitable biologically compatible aqueous buffer to create a solution having a PEG concentration of about 1 to about 20% (w/v) and a α-cyclodextrin concentration of about 0.1 to about 10% (w/v); c) mixing the solution of b) for a sufficient time to provide an inclusion step in which poly(ethylene glycol) (PEG) polymers or derivatives thereof and cyclodextrin molecules obtain a polyrotaxane-like configuration in which the poly(ethylene glycol) (PEG) polymers or derivatives thereof are included in the cavity of each of α-cyclodextrin molecule in a skewered manner; d) adding a photoinitiator to the solution of c) to create a final concentration of photoinitiator of between about 0.01 to about 0.1% (w/v); e) exposing the solution of d) to electromagnetic radiation at a wavelength specific to the photoinitiator for a sufficient amount of time to initiate the polymerization of the polymers in the solution; and f) allowing the polymerization to complete.

In accordance with another embodiment, the present invention provides a method for making a 2-dimensional cell-encapsulated hydrogel biomaterial comprising: a) obtaining a solution of α-cyclodextrin molecules in a suitable biologically compatible aqueous buffer and placing it in a shallow dish or container or similar support; b) adding to a) a sufficient amount of poly(ethylene glycol) (PEG) polymers or derivatives thereof in a suitable biologically compatible aqueous buffer to create a solution having a PEG concentration of about 1 to about 20% (w/v) and a α-cyclodextrin concentration of about 0.1 to about 10% (w/v); c) mixing the solution of b) for a sufficient time to provide an inclusion step in which poly(ethylene glycol) (PEG) polymers or derivatives thereof and α-cyclodextrin molecules obtain a polyrotaxane-like configuration in which the poly(ethylene glycol) (PEG) polymers or derivatives thereof are included in the cavity of each of α-cyclodextrin molecule in a skewered manner; d) adding a photoinitiator to the solution of c) to create a final concentration of photoinitiator of between about 0.01 to about 0.1% (w/v); e) exposing the solution of d) to electromagnetic radiation at a wavelength specific to the photoinitiator for a sufficient amount of time to initiate the polymerization of the polymers in the solution; f) soaking the polymerized gel of e) for a sufficient period of time to remove any α-cyclodextrin which do not have the poly(ethylene glycol) (PEG) polymers or derivatives thereof are included in their cavities; and g) seeding a quantity of cells onto the polymerized gel of f) at a density of between about 5000 to about 50,000 cells/cm² in a biologically compatible growth media.

In accordance with still another embodiment, the present invention provides a method for making a 3-dimensional cell-encapsulated hydrogel biomaterial comprising: a) obtaining a solution of α-cyclodextrin molecules in a suitable biologically compatible aqueous buffer and placing it in a container or similar support; b) adding to a) a sufficient amount of poly(ethylene glycol) (PEG) polymers or derivatives thereof in a suitable biologically compatible aqueous buffer to create a solution having a PEG concentration of about 1 to about 20% (w/v) and a α-cyclodextrin concentration of about 0.1 to about 10% (w/v); c) mixing the solution of b) for a sufficient time to provide an inclusion step in which poly(ethylene glycol) (PEG) polymers or derivatives thereof and α-cyclodextrin molecules obtain a polyrotaxane-like configuration in which the poly(ethylene glycol) (PEG) polymers or derivatives thereof are included in the cavity of each of α-cyclodextrin molecule in a skewered manner; d) adding a photoinitiator to the solution of c) to create a final concentration of photoinitiator of between about 0.01 to about 0.1% (w/v); e) seeding a quantity of cells into the solution of d) at a quantity of between about 500,000 to about 5×10⁶ cells in a biologically compatible growth media; and f) exposing the solution of e) to electromagnetic radiation at a wavelength specific to the photoinitiator for a sufficient amount of time to initiate the polymerization of the polymers in the solution.

In accordance with an embodiment, the present invention provides multifunctional biomaterials which can be electrospun into multifunctional nanofibers. The nanofibers of the present invention were developed based on the IC of aliphatic polyester-α-cyclodextrin (e.g., PCL-α-CD) for tissue engineering applications. However, one of ordinary skill would understand that any aliphatic or hydrophobic biocompatible polymer would be suitable.

The term “electrospinning” is known in the art, and is a process in which a charged polymer jet is collected on a grounded collector; a rapidly rotating collector results in aligned nanofibers while stationary collectors result in randomly oriented fiber mats. The polymer jet is formed when an applied electrostatic charge overcomes the surface tension of the solution. There is a minimum concentration for a given polymer, termed the critical entanglement concentration, below which a stable jet cannot be achieved and no nanofibers will form—although nanoparticles may be achieved (electrospray). A stable jet has two domains, a streaming segment and a whipping segment. While the whipping jet is usually invisible to the naked eye, the streaming segment is often visible under appropriate lighting conditions. Observing the length, thickness, consistency and movement of the stream is useful to predict the alignment and morphology of the nanofibers being formed. The stream can be optimized by adjusting the composition of the solution and the configuration of the electrospinning apparatus, thus optimizing the alignment and morphology of the fibers being produced. Any known methods for electrospinning the polymers used herein can be used with the methods of the present invention to provide the multifunctional biomaterials disclosed herein.

It will be understood by those of ordinary skill that the multifunctional nanofiber materials can be conjugated with many different types of compounds or molecules, through the substitution of the hydroxyls on the α-CD molecules in the biomaterials. In accordance with an embodiment, the nanofibers can be conjugated to fluorescent dyes, peptides, small molecules and other biologically active compounds. In accordance with another embodiment, the multifunctional nanofiber materials can be conjugated with polystyrene nanobeads.

Methods of making the multifunctional nanofiber materials of the present invention are also provided herein. In an embodiment, the present invention provides a method for making a multifunctional biomaterial comprising: a) obtaining a sufficient amount of hydrophobic biocompatible polymers or derivatives thereof in a suitable organic solvent to create a solution having a polymer concentration of about 0.1 to about 0.2 g/mL polymer and heating the solution to about 45° C. to 60° C.; b) adding to a) a solution of α-cyclodextrin molecules in a suitable polar aprotic solvent at a concentration of about 0.4 to 0.6 g/ml to create a mixture with a final concentration of α-cyclodextrin molecules in the mixture of between about 0.005 to about 0.008 g/ml; c) mixing the solution of b) for a sufficient time to provide an inclusion step in which the hydrophobic polymers or derivatives thereof and cyclodextrin molecules obtain a pseudopolyrotaxane configuration in which the polymers or derivatives thereof are included in the cavity of each of α-cyclodextrin molecule in a skewered manner; d) cooling the mixture of c) to room temperature; e) evaporating the organic solvent away from mixture of d) to produce a dried product; and f) washing the product of e) with water to remove excess α-cyclodextrin molecules.

In one or more embodiments, the hydrophobic polymer is PCL, the organic solvent is acetone, and the polar aprotic solvent is DMF.

In accordance with another embodiment, the inventive method further comprises g) dissolving the product of e) in a mixture of dichloromethane and DMSO to create a solution having a concentration between about 5% to about 15% w/v of polymer product; and h) electrospinning the solution to create one or more nanofibers and allowing the fibers to dry.

### EXAMPLES

Synthesis of an aldehyde substituted α-CD or α-CDCHO. Dess-Martin periodinane (DMP) (0.9 g, 2 mmol) was added to a solution of α-CD (1.0 g, 1 mmol) in anhydrous dimethyl sulfoxide (DMSO) (5.0 mL), and stirred for 18 hours. After centrifugation, the supernatant was precipitated in acetone (200 mL) and washed twice with CH₂Cl₂ (20 mL). A white product (0.65 g) with yield ˜65% was obtained after drying in vacuo. ¹H NMR (D₂O: 4.79 ppm): δ 3.50-4.10, 4.62, 5.0-5.05, 5.09-5.60. ¹³C NMR (D₂O): δ 60.9, 72.3-72.6, 73.8-73.9, 82.7, 83.4, 87.9, 102.0. Mass (MALDI-TOF): 966+23 [Na⁺], 968+23 [Na⁺], and 970+23 [Na⁺]; 966+17+23 [Na+], 968+17+23 [Na+], and 970+17+23 [Na+] (OH from hydrated state of aldehyde).

Synthesis of a carboxylic acid substituted α-CD or α-CDCOOH. Potassium peroxymonosulfate, or oxone (0.35 g, 2.25 mmol), was added to a solution of α-CDCHO (0.37 g, 0.37 mmol) in anhydrous N,N′-dimethylformamide (DMF) (6.0 mL), and vigorously stirred for 19 hours. After centrifugation, the supernatant was filtered through a filter (0.2 μm) and precipitated in acetone (300 mL). The product was dissolved in a minimum amount of water and reprecipitated in acetone (200 mL). The product was further purified by a Sephadex column chromatography (G10, GE Healthcare Biosciences). A white product (0.24 g) with yield ˜64% was obtained after drying in vacuo. ¹H NMR (D2O: 4.79 ppm): δ 3.61, 3.87, 3.94, 4.23, 4.62, 5.10, 5.43. ¹³C NMR (D2O): δ71.0-75.0, 82.5-83.0, 87.2, 101.2-102.4, 175.2. Mass (MALDI-TOF): 986+23 [Na+], 1000+23 [Na+], and 1014+23 [Na+].

Synthesis of an amine substituted α-CD or α-CDNH₂. α-CDNH₂ was synthesized in a two-step process. In the first step, N, N′-carbonyldiimidazole (CDI) (0.33 g, 0.3 mmol) was added to a solution of α-CD (2.0 g, 2.0 mmol) in anhydrous DMF (6.0 mL). After 2 hours of stirring, the product was precipitated thrice in acetone (200 mL). After vacuum drying overnight, this activated α-CD (1.93 g, 1.8 mmol) was dissolved in ethylenediamine (5 mL) and stirred overnight. The product was precipitated in acetone (200 mL), filtered and washed again with acetone (100 mL). The product was dissolved in a minimum amount of water and reprecipitated twice in acetone (200 mL). A white powder (1.75 g) with 90% yield was obtained after vacuum drying. For cell culture experiments, α-CDNH₂ was further purified by a Sephadex column chromatography (G10, GE Healthcare Biosciences). A white product (0.24 g) with yield ˜64% was obtained after drying in vacuo. ¹H NMR (D₂O: 4.79 ppm): δ 2.67, 2.76, 2.93, 3.50-3.63, 3.74-3.83, 3.85, 3.92, 4.24, 4.48, 5.02. ¹³C NMR (D₂O): δ 42.1, 44.6, 46.5, 50.5, 62.0, 74.6-75.6, 83.3, 104.0, 160.0. Mass (MALDI-TOF): 1058+23 [Na⁺].

Synthesis of β-CDCHO. To a solution of β-CD (1.0 g, 0.9 mmol) in anhydrous DMSO (5.0 mL), DMP (0.81 g, 1.8 mmol) was added and stirred for 18 hours. After centrifugation, the supernatant was precipitated in acetone (200 mL) and washed twice with CH₂Cl₂ (20 mL). A white product (0.67 g) with yield ˜60% was obtained after drying in a vacuum. ¹H NMR (D₂O: 4.79 ppm): 3.45-3.70, 3.75-4.2, 4.65, 5.0-5.25, 5.25-5.70. Mass (MALDI-TOF): 1129+18 [H₂O], 1131+18 [H₂O], and 1133+18 [H₂O].

Synthesis of β-CDCOOH. To a solution of β-CDCHO (0.30 g, 0.26 mmol) in anhydrous DMF (2.0 mL), potassium peroxymonosulfate (Oxone) (0.36 g, 2.36 mmol) was added. The solution was vigorously stirred for 18 hours. After centrifugation, the supernatant was filtered through 0.2 μm and precipitated in acetone (300 mL). The product was dissolved in a minimum amount of water and reprecipitated in acetone (200 mL). The product was further purified by a Sephadex column chromatography (G10, GE Healthcare Biosciences). A white product (0.21 g) with yield ˜62% was obtained after drying in a vacuum. ¹H NMR (DMSO-D₆: 2.54 ppm): 3.20-3.45, 3.90-4.10, 4.40-4.50, 4.70-4.90, 5.50-5.90. ¹³C NMR (D₂O): 60.2-61.2, 72.3-73.9, 81.5-83.0, 87.9, 100.4-102.5, 172.6. Mass (MALDI-TOF): 1161+18 [H₂O], 1163+18 [H₂O], and 1165+18 [H₂O].

Preparation of 2D and 3D hydrogels. PEG diacrylate (PEGDA) (Mw ˜3400 Da, PDI 1.1 from SunBio Inc.) in PBS (pH 7.4) was added to saturated phosphate buffered saline (PBS) solutions of α-CD (Sigma-Aldrich) and α-CD-derivatives (α-CDCOOH, α-CDCHO and α-CDNH2) to make solutions with final PEGDA concentrations of 5%, 10% and 15% (w/v), and final α-CD (derivatives) concentrations of 0.5%, 1% and 5% (w/v). After vortexing for a few minutes, a photoinitiator solution (Irgacure® 2959 [(Ciba specialty chemical now BASF Resins] in 70% ethanol) was added to these solutions to make a final initiator concentration of 0.05% (w/v). A perfusion chamber (diameter 9.0 mm, height 1.0 mm, Grace Bio-Labs) on a microscope glass slide and an Eppendorf tube cap (0.5 mL) were taken as molds for 2D hydrogels and 3D hydrogels, respectively. To make gels, the pre-gel solutions were exposed to UV light (wavelength-365 nm) for 5 minutes. On 2D hydrogel surfaces, hMSCs were seeded with a cell density of 20,000 cells/cm². As an example, 40 μL, of PEGDA (10%, w/v) solution was added to a 9.0 mm diameter perfusion chamber and polymerized under UV for 5 minutes. Before seeding cells, the 2D hydrogel was soaked overnight in PBS (pH 7.4) to remove any unthreaded α-CD. For 3D hydrogels, 2 million hMSCs were added to the 100 μL pre-gel solution and photopolymerized for 5 minutes.

Histochemistry PEG α-CD. Harvested constructs were fixed for 24 hours in 4% paraformaldehyde at 4° C. and then stored in 70% ethanol until processing. The constructs were then dehydrated in a sequential series of ethanol solutions (i.e., 80%, 95% and 100%) and 100% xylene, and embedded in paraffin overnight at 60° C. The paraffin block was sliced into 5 μm sections, mounted onto microscope slides and incubated on a 40° C. plate for at least 1 hour. Prior to staining, samples were de-waxed and rehydrated immediately before staining. Safranin-O/Fast green staining was used for detecting proteoglycans content. H & E staining was performed for studying cell morphology (data not shown).

F-actin staining PEG α-CD. The 2D samples were rinsed thrice with PBS, fixed with 4% paraformaldehyde for 10 minutes, and treated with 0.1% Triton™ X-100 for 5 minutes at room temperature. After rinsing samples twice with PBS (pH 7.4), 2.5% (v/v) Texas Red®-X phalloidin (Invitrogen™, Life Technologies) and 4 μM Hoechst 33258 solutions were added, and the samples were kept in the dark for 30 minutes. After washing with PBS three times, images were taken with Nikon DXM1200 or Zeiss Axio optical microscopes. The images were merged and analyzed using ImageJ (US National Institutes of Health).

Live/dead staining PEG α-CD. Viability analysis of the 3D encapsulated cells was performed using manufacturer's guidelines for the LIVE/DEAD® Viability/Cytotoxicity Kit (Invitrogen™, Life Technologies). The samples were rinsed thrice with PBS, and thin sections (<300 μm) were incubated in live-dead medium for 30 minutes at 37° C. The medium contained Dulbecco's Modified Eagle Medium (DMEM), 4 mM calcein AM and 4 mM ethidium homodimer-1. After washing thrice with PBS, images were taken with a Nikon DXM1200 fluorescence microscope with an optical filter (485±10 nm) for calcein AM (live cells) and a (530±12 nm) optical filter for ethidium homodimer-1 (dead cells). The live/dead cells images were merged and analyze using ImageJ (U.S. National Institutes of Health).

Compression modulus and swelling ratio measurements PEG α-CD. The hydrogels' moduli were measured using an Electroforce 3200 testing instrument (Bose Corp.). Data were collected by compressing cylindrical gels from 0% to 10% strain at 0.44% per second. The modulus was calculated by best-curve fit in the linear region of the stress vs. strain plot. The hydrogels were incubated in PBS (pH 7.4) for 48 hours followed by measuring their wet and dry weights. The ratio of wet weight over dry weight was taken as the swelling ratio of the hydrogels.

Biochemical Assay PEG α-CD. The dried constructs were crushed with a tissue grinder (pellet pestle mixer; Kimble/Kontes) and digested in 1 mL of papainase solution (papain, 125 mg/mL; Worthington Biomedical), 100 mM phosphate buffer, 10 mM cysteine, 10 mM EDTA, pH 6.3) for 18 hours at 60° C. The DNA content was determined using Hoechst 33258 dye on a fluorometer with calf thymus DNA solution (0-400 ng/mL) as standards, as previously described (Anal. Biochem., 1988; 174:168-76). GAG content was measured using a dimethylmethylene blue dye-binding assay with chondroitin sulfate solution (0-50 μg/mL) as standards, as previously described (Biochim. Biophys. Acta, 1986; 883:173-7). Total collagen content was determined by measuring the hydroxyproline content according to the method described by Stegemann and Stalder with hydroxyproline solution (0-5.0 μg/mL) as standards, using 0.1 as the mass ratio of hydroxyproline to collagen. Briefly, the papain-digested solution was acid-hydrolyzed with 6 M HCl at 115° C. for 18 hours, neutralized by 2.5 M NaOH and treated with chloramine-T/p-dimethyl aminobenzaldehyde. The absorbance at 557 nm was measured to determine collagen content. DNA, GAG and total collagen content were normalized to the dry weight of the respective construct (μg/mg). In addition, the GAG and total collagen content were also normalized to the DNA content of the respective construct (μg/μg).

Characterization techniques PEG α-CD. The functionalized α-CDs were analyzed by MALDI-TOF spectrometry (Voyager DE-STR, Applied Biosystems®, Life Technologies). ¹H and ¹³C NMR experiments were performed on either Bruker (Billerica) 300 MHz or 400 MHz nuclear magnetic resonance (NMR) spectrometers. The iNMR processing software (inmr.net) was used for processing the spectrum.

Cell culture PEG α-CD. hMSCs were obtained as a generous gift from Arnold Caplan, Case Western Reserve University. MSCs were cultured in expansion media on 2D surfaces, while in cell-differentiation media in 3D gels. All constructs and substrates were cultured at 37° C. with 5% CO₂, and the media were changed every 2 to 3 days until harvesting. The expansion medium consists of DMEM (high glucose, 1×), fetal bovine serum (FBS, 10%), penicillin/streptomycin (1%, v/v), glutamax (1%, v/v) and basic fibroblast growth factor (bFGF, 8 ng/mL). The chondrogenic differentiation medium consists of DMEM (high glucose, lx), FBS (10%, v/v), dexamethasone (100 nM), penicillin/streptomycin (1%, v/v), sodium pyruvate (100 ug/mL), L-proline (40 ug/mL), ascorbic acid-2-phosphate (50 ug/mL), insulin, transferrin, selenous acid (ITS) (1% v/v).

Gene expression PEG α-CD. Cellular mRNAs were extracted as previously described by Strehin et al. (Methods Mol. Biol., 2009; 522:349-62). Briefly, hydrogels were put into 1.5 mL Rnase-free Eppendof tubes, soaked in 1 mL Trizol solution and crushed with an Rnase-free pestle. RNAs were exacted according to the manufacture's manual for Trizol, and then precipitated, washed with isopropanol and 75% ethanol, and redissolved in diethylpyrocarbonate- (DEPC-) treated water. This solution of mRNA was incubated at 60° C. for 10 minutes and quickly put on ice. The concentration of mRNA was quantified using a Nanodrop™ 2000 spectrophotometer (Thermo Scientific). The cDNA was synthesized according to the manufacturer's protocol for the Superscript 1st Strand System Kit (Invitrogen™, Life Technologies). One microgram cDNA per sample was used for real-time polymerase chain reaction (PCR) with SYBR® Green PCR Master Mix (Applied Biosystems®, Life Technologies) using the primers shown in Table 1 with β-actin as a reference gene. The level of expression was calculated using the Pfaffl method (Nucleic Acids Res., 2001; 29:e45).

TABLE 1
Primers for PCR analysis PEG α-CD

| | | Annealing |
|---|---|---|
| Gene | Sequence (Forward and Reverse) | Temperature |
| aggrecan | 5'-TGGGAACCAGCCTATACCCCAG-3' | 60° C. |
| | (SEQ ID NO: 1) | |
| | 5'-CAGTTGCAGAAGGGCCTTCTGTA | |
| | C-3'(SEQ ID NO: 2) | |
| collagen | 5'-GGAATGCCTGTGTCTGCTTT-3' | 60° C. |
| type X | (SEQ ID NO: 3) | |
| | 5'-TGGGTCATAATGCTGTTGCC-3' | |
| | (SEQ ID NO: 4) | |
| collagen | 5'-CGCCGCTGTCCTTCGGTGTC-3' | 60° C. |
| type II | (SEQ ID NO: 5) | |
| | 5'-AGGGCTCCGGCTTCCACACAT-3' | |
| | (SEQ ID NO: 6) | |
| Sox 9 | 5'-GCATGAGCGAGGTGCACTC-3' | 60° C. |
| | (SEQ ID NO: 7) | |
| | 5'-TCTCGCTTCAGGTCAGCCTTG-3' | |
| | (SEQ ID NO: 8) | |
| β-actin | 5'-GCTCCTCCTGAGCGCAAGTAC-3' | 60° C. |
| | (SEQ ID NO: 9) | |
| | 5'-GGACTCGTCATACTCCTGCTTGC-3' | |
| | (SEQ ID NO: 10) | |

Statistical Analysis PEG α-CD. One-way ANOVA was used to detect significant effects among groups. Tukey's multiple comparison tests were used to detect any significant differences between groups, and a p-value ≦0.05 was considered significant. The error bars displayed for the gene-expression data showed the calculated maximum (RQMax) and minimum (RQMin) expression levels that represent the standard deviation of expression level (RQ value).

Characterization techniques PEG α-CD. Threading of α-CDNH₂ onto PEGDA chains was determined qualitatively by a ninhydrin assay (Anal. Biochem., 2001; 292:125-9). In brief, hydrogels were washed rigorously with deionized water and lyophilized. The known amount of the dried hydrogel was hydrolyzed overnight at 115° C. with 6 N HCl, followed by neutralization with NaOH. The aliquot was mixed with ninhydrin reagent and kept at 110° C. for 10 minutes. After cooling down to room temperature, the color of the solution was analyzed. The hydrogels with amine functional groups turned purple. The nitrogen contents in PEGDA/α-CDNH₂ hydrogels were further determined by X-ray photoelectron spectroscopy (XPS) (PHI 5400 XPS, Perkin-Elmer). The Fourier transform infrared-attenuated total reflectance (FTIR-ATR) spectroscopy (Bruker, Vector 22 with a Pike Miracle ATR attachment) was performed on dried hydrogel surfaces.

Cell-responsive hydrogels—conjugation of YRGDS to α-CDNH₂ via suberic acid bis(N-hydroxysuccinimide ester) linker: a PEGDA solution (20 μL of 520 mg in 2600 μL of PBS, pH 7.4) was added to an α-CDNH₂ solution (2 μL of 20 mg in 200 μL of PBS, pH 7.4) and mixed for ˜10 minutes. To this solution was added and mixed, 1 μL of YRGDS (Biomatik Corp.) solution (9 mg in 90 μL of PBS, pH 7.4) and 0.7 μL of suberic acid bis(N-hydroxysuccinimide ester) (12 mg in 120 μL of DMSO, Sigma-Aldrich). This solution was diluted to 40 μL by adding 17 μL of PBS (pH 7.4) to make a pre-gel solution of 5% PEGDA (w/v). Similarly, for 10% and 15% PEGDA (w/v) pre-gel solutions, respective amounts of PEGDA stock solution were added, while keeping amounts of α-CDNH₂ and suberic acid bis(N-hydroxysuccinimide ester) unchanged. After adding Irgacure solution (70% ethanol, final concentration, 0.5% [v/v]), theses pre-gel solutions were polymerized under UV light (365 nm for 5 minutes, ˜5.0 mW/cm²) in perfusion chambers (Grace-BioLabs, Inc., diameter 9 mm, height 1 mm, volume-40 μL). The gels were soaked in PBS overnight to remove DMSO and unreacted components prior to culturing cells on the top surfaces of the hydrogels.

Cell-responsive hydrogels—conjugation of YRGDS via α-CDNHS: First, α-CDNHS was synthesized as in the following example. After stirring a mixture of α-CDCOOH (50 mg), 1-ethyl-3-(3-dimethylaminopropyl)carbodiimide (EDC) (30 mg), and N-hydroxysuccinimide (NHS) (25 mg) in PBS (pH 7.4, total 500 μL) for 30 minutes at room temperature, the product was precipitated in acetone (2 mL). The precipitate was dissolved in DMSO (200 μL), filtered through a filter (0.2 μm pore size) and reprecipitated in acetone (1 mL). After precipitating twice in acetone from DMSO, the product was vacuum dried to yield a white powder (32 mg, ˜55%). Second, α-CDNHS was threaded onto PEG chains followed by conjugating it with YRGDS and preparing hydrogels. As an example, YRGDS (0.32 mg) was added to a mixture of α-CDNHS (0.63 mg) and PEGDA in PBS (10 mg, 100 μL) and vortexed. Hydrogels were synthesized using this solution in a similar procedure as mentioned earlier.

Synthesis of PCL-α-CD IC. PCL (1.0 g, Mw 70 k˜90 k Da; Sigma-Aldrich) was dissolved in acetone (60 mL) and heated at 50° C. in a silicon oil bath. α-CD (0.5 g, Sigma-Aldrich) was dissolved in 10 mL of dimethylformamide (DMF) and added dropwise to the heated PCL-acetone solution. After stirring for 2 hours, the mixture was air-cooled to room temperature. This solution was poured into a glass flat-bottom PYREX® container (Corning Inc. Life Sciences) and stirred slowly overnight at room temperature to evaporate the acetone. A thin layer of PCL-α-CD film was formed, which was soaked in and washed multiple times with water to remove any unthreaded α-CD. PCL-α-CD IC was further characterized by 1H-NMR (300 or 400 MHz; Bruker), wide-angle X-ray diffraction (WAXD) from 20=5° to 35° (PANalytical MPD Pro Diffractometer, Cu-Kα radiation; PANalytical B.V.) and Fourier transform infrared-attenuated total reflectance (FTIR-ATR) (Bruker, Vector 22 with a Pike Miracle ATR attachment) spectroscopy within a range of wavenumber 700-3800 cm-1. WAXD and FTIR-ATR were also performed on PCL only and α-CD only samples, as controls.

Electrospinning of PCL and PCL-α-CD IC nanofibers. PCL was dissolved in a mixture of dichloromethane (DCM) and dimethyl sulfoxide (DMSO) (17/9, v/v) at a concentration of 10% (w/v). The solution was drawn into a 1 mL syringe (Norm-Ject, Henke-Sass Wolf GmbH) with a 30 G needle (Becton, Dickinson and Co.) and electrospun at 8 kV and 5 mL/h PCL-α-CD was dissolved in a mixed solvent of DCM and DMSO (2/3, v/v) at a concentration of 10% (w/v) and filled into the same kind of syringe and needle. The PCL-α-CD fibers (605±85 nm, n=100 nm) were electrospun at 5.5 kV and 6 mL/h to obtain fibers with similar diameter to those of PCL (617±170 nm, n=100). Fibers were collected onto aluminum foil covered with 15 mm diameter microscope cover slips (Thermo Fisher Scientific), which were kept at a distance of 16 cm from the tip of the syringe needle. Electrospun fibers-covered cover slips were cut off from the aluminum foil and kept for further use. Before seeding with cells, fiber samples were put into 24-well plates and sterilized by overnight UV exposure.

Fluorescamine conjugation to PCL-α-CD IC fibers. PCL and PCL-α-CD fibers on microscope cover slips (dia ˜15 mm) were soaked in DMSO containing N,N′-carbonyldiimidazole (N,N′-CDI; Sigma-Aldrich) at room temperature. Ethylenediamine (Sigma-Aldrich) was added to these fibers, and after 30 minutes of shaking, both fibers were taken out, washed with fresh DMSO and soaked in fluorescamine-DMSO solution. After subsequent washing with fresh DMSO and water, fluorescence images of two different samples were taken on a Nikon DXM1200 microscope under both bright field and UV light.

Polystyrene nanobead conjugation to PCL-α-CD IC fibers. PCL and PCL-α-CD fibers were soaked in N,N′-CDI/DMSO solution while undergoing shaking. After 1 hours both fibers were taken out, washed with fresh DMSO and soaked in DMSO containing polystyrene nanobeads with amine functional groups (0.2 μm dia; Invitrogen™, Life Technologies). After shaking for ˜4 hours, fibers were washed with ethanol to remove any unconjugated nanobeads that had settled on the fiber surface. The fibers on cover slips were placed vertically in both DMSO and ethanol to avoid any gravitational settling or physical adsorption of beads on the fibers. These fibers were vacuum dried, sputter coated (Anatech Hummer 6.2) with platinum and characterized by SEM (FEI Quanta 200).

Cell culture of PCL α-CD on nanofibers. Human adipose-derived stem cells (hADSCs) were isolated as previously described (Stem Cells, 24, 376-385(2006)), received via a material transfer agreement, and expanded up to passage 4 before usage. For expansion, cells were cultured in a medium consisting of low glucose (1.0 g/L) DMEM supplemented with 876 mg/L of L-glutamine, 10% fetal bovine serum (FBS), 100,000 U/L penicillin, 10 mg/L streptomycin and 1 μg/L basic fibroblast growth factor (Invitrogen™, Life Technologies). For osteogenic induction, cells were seeded onto nanofibers at a cell density of 5,000/cm² in an osteogenic medium composed of high glucose (4.5 g/L) DMEM supplemented with 100,000 U/L penicillin, 10 mg/L streptomycin, 10% FBS, 50 μM ascorbic acid, 0.1 μM dexamethasone and 10 mM glycerol-2-phosphate disodium salt. Cells were harvested and analyzed on days 7, 14 and 21.

Gene expression PCL α-CD. Cellular mRNAs were extracted as previously described by Strehin et al.₆₀ Briefly, the mRNA was extracted with 1 mL trizol per well and then precipitated, washed with isopropanol and 75% ethanol, and redissolved in diethylpyrocarbonate- (DEPC-) treated water. This solution of mRNA was incubated at 60° C. for 10 min and quickly put on ice. The concentration of mRNA was quantified using a Nanodrop™ 2000 spectrophotometer (Thermo Scientific). The cDNA was synthesized according to the manufacturer's protocol for the Superscript 1^{st} Strand System Kit (Invitrogen™, Life Technologies). The cDNA was used for real-time polymerase chain reaction (PCR) with SYBR® Green PCR Master Mix (Applied Biosystems, Life Technologies) using the primers shown in Table 1 with β-actin as a reference gene. The level of expression was calculated using the Pfaffl method.

Biochemical assays PCL α-CD. Biochemical assays were performed using a revised version of the method described by Strehin et al. Briefly, after aspirating off media, samples were rinsed thrice with PBS, removed from the 24-well plate and lyophilized. After measuring the dry weight of the samples, they were incubated overnight at 60° C. in 500 μL papainase buffer, which contained 1 M Na₂HPO₄, 10 mM disodium EDTA.2H₂O, 10 M L-cysteine and 9.3 units/mL papain type III (Worthington Biochemical Corp.). Supernatants were collected after centrifugation and used for DNA and collagen assays.

For DNA assays, 30 μL of sample digest was mixed with 3 mL of pH 7.4 DNA buffer solution, which contained 100 μg/mL Hoechst 33258, 10 mM Tris base, 200 mM NaCl and 1 mM disodium EDTA.2H₂O. The mixture was then analyzed with a DyNA Quant 200 Fluorometer (Hoefer, Inc.), with an excitation/emission of 365/460 nm. The measurements were analyzed with a calibration curve using DNA solutions made with calf thymus DNA (Invitrogen™, Life Technologies).

TABLE 2
Primer sequences for real-time PCR for PCL α-CD

| | | Annealing |
|---|---|---|
| Gene | Sequence (Forward and Reverse) | Temperature |
| Collagen type I | 5'-GCCAAGAGGAAGGCCAAGTC-3' | 60° C. |
| | (SEQ ID NO: 11) | |
| | 5'-AGGGCTCGGGTTTCCACAC-3' | |
| | (SEQ ID NO: 12) | |
| collagen type X | 5'-GGAATGCCTGTGTCTGCTTT-3' | 60° C. |
| | (SEQ ID NO: 3) | |
| | 5'-TGGGTCATAATGCTGTTGCC-3' | |
| | (SEQ ID NO: 4) | |
| Osteopontin | 5'-GACACATATGATGGCCGAGGTGATAG-3' | 60° C. |
| | (SEQ ID NO: 13) | |
| | 5'-GGTGATGTCCTCGTCTGTAGCATC-3' | |
| | (SEQ ID NO: 14) | |
| Runx2 | 5'-CTTCACAAATCCTCCCCAAGTAGCTACC-3' | 60° C. |
| | (SEQ ID NO: 15) | |
| | 5'-GGTTTAGAGTCATCAAGCTTCTGTCTGTG-3' | |
| | (SEQ ID NO: 16) | |
| β-actin | 5'-GCTCCTCCTGAGCGCAAGTAC-3' | 60° C. |
| | (SEQ ID NO: 9) | |
| | 5'-GGACTCGTCATACTCCTGCTTGC-3' | |
| | (SEQ ID NO: 10 | |

For collagen assays, 100 μL of papain digest was added to 100 μL of 37% (v/v) conc. HCl and the mixture was hydrolyzed at 115° C. for 18 hours. Samples were neutralized with aq. NaOH and the volume was brought up to 3.5 mL with deionized water. Added to this solution (1 mL) was 0.5 mL of chloramine-T solution (69 mM chloramine-T in 89% [v/v] pH 6 buffer and 11% [v/v] isopropanol); it was maintained at room temperature for 20 minutes. The pH 6.0 buffer solution contained 0.57 M NaOH, 0.16 M citric acid monohydrate, 0.59 M sodium acetate trihydrate, 0.8% (v/v) glacial acetic acid, 20% (v/v) isopropanol, 79.2% (v/v) dd H₂O and 5 drops of toluene. Added to this solution was 0.5 mL of 4-(dimethylamino)benzaldehyde (pDAB) (1.17 MpDAB in 70% [v/v] isopropanol, 30% [v/v] of 60% perchloric acid in water); and it was incubated at 60° C. for 30 minutes. After cooling to room temperature, the samples were analyzed for their absorbance at 557 nm using a DU500 UV-Vis spectrophotometer (Beckman Coulter, Inc) and compared to a standard solution of hydroxyproline.

Live/dead staining PCL α-CD. Cells seeded on both fibers were stained with the LIVE/DEAD® Viability/Cytotoxicity Kit (Invitrogen™, Life Technologies) as per the manufacturer's protocol. Briefly, DMEM supplemented with 4 μM calcein-AM, 4 μM ethidium homodimer-1 and 4 μM Hoechst 33258 was added to cells and incubated at 37° C. and 5% CO₂ for 30 minutes. After rinsing the samples thrice with PBS, fluorescent images were taken with a Zeiss Axio optical microscope (HXP 120 fluorescent illuminator) (Carl Zeiss Microscopy). ImageJ (U.S. National Institutes of Health) was used to merge images for further analysis.

F-actin staining PCL α-CD. Cells were fixed with 4% paraformaldehyde for 10 minutes and incubated with 0.1% Triton™ X-100 at room temperature for 5 minutes. Subsequently, cells were rinsed with PBS, before adding 2.5% (v/v) Texas Red-X® phalloidin (200 U/mL; Invitrogen™, Life Technologies) solution containing 4 μM Hoechst 33258. After being maintained in the dark for 30 min, samples were rinsed thrice with PBS. Images of these stained cells were taken with a Zeiss Axio optical microscope, merged and analyzed using ImageJ.

Alizarin Red Staining PCL α-CD. The samples were rinsed twice with PBS after carefully aspirating off media from each well. Cells were fixed with 4% paraformaldehyde at room temperature for 15 minutes and rinsed thrice with deionized water. Subsequently, 1 mL of 40 mM alizarin red S solution (pH 4.1) was added to each well. Dye was aspirated off after 20 min, and fibers were rinsed thrice with distilled water. Images of the wells were taken with an Olympus C-765 camera (Olympus America).

Alkaline Phosphatase (ALP) Staining PCL α-CD. Cells were rinsed with Tyrode's balanced salt solution (TBSS, Sigma-Aldrich) twice, and fixed with a citrate-buffer acetone solution for 30 seconds. The citrate-buffer acetone solution was composed of a 60% (v/v) citrate working solution and 40% (v/v) acetone. The citrate working solution was made by adding 2 mL of citrate concentrated solution (Sigma-Aldrich) to 98 mL of water. Cells were rinsed twice with PBS after removing the salt solution. One mL of fast violet-naphthol solution was added to each well, and incubated in the dark for 45 minutes at room temperature. Fast violet-naphthol solution was made by adding 0.5 mL of naphthol AS-MX alkaline solution (Sigma-Aldrich) to 12 mL of fast violet solution, which was made by dissolving one capsule of fast violet (Sigma-Aldrich) in 48 mL of water. Images of the stained cells were taken with an Olympus C-765 camera.

Statistical analysis PCL α-CD. Data are expressed as mean±standard deviation. Statistical analysis was performed using SPSS v.19 (IBM Corp.). One-way ANOVA was performed among groups to determine any statistically significant differences in values of means. Samples with equal variances and sizes were analyzed using Tukey's post-hoc test, while the Games-Howell post-hoc test was used for samples with unequal variances and unequal sample sizes. P≦0.05 was considered statistically significant.

### Example 1

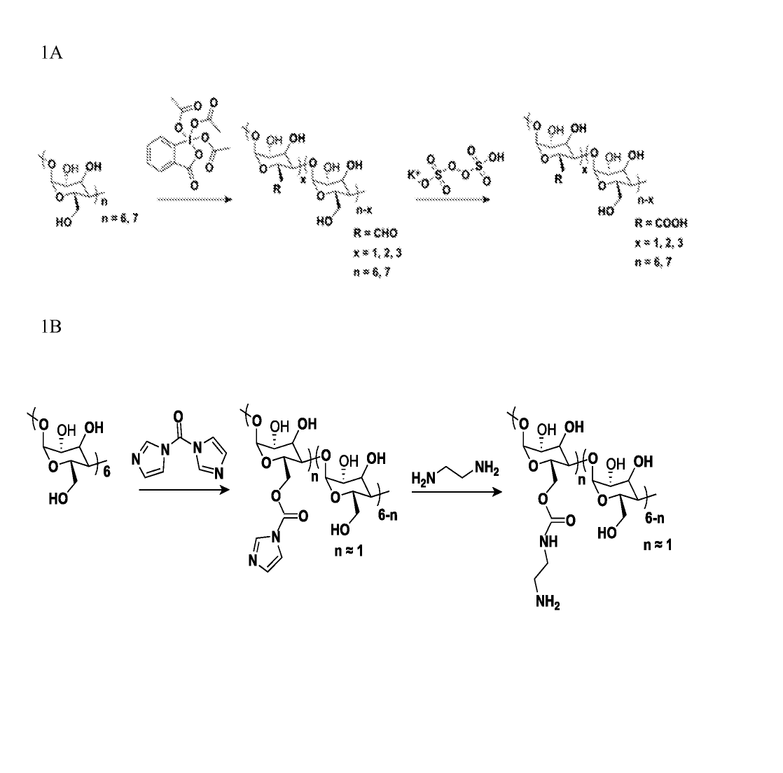

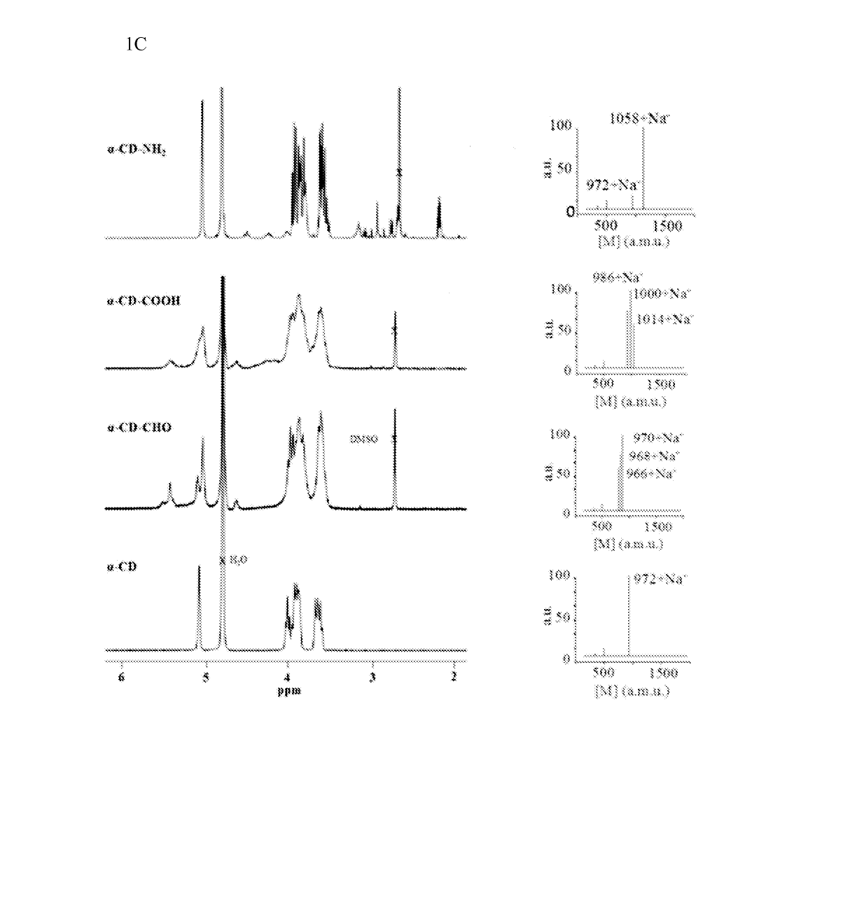

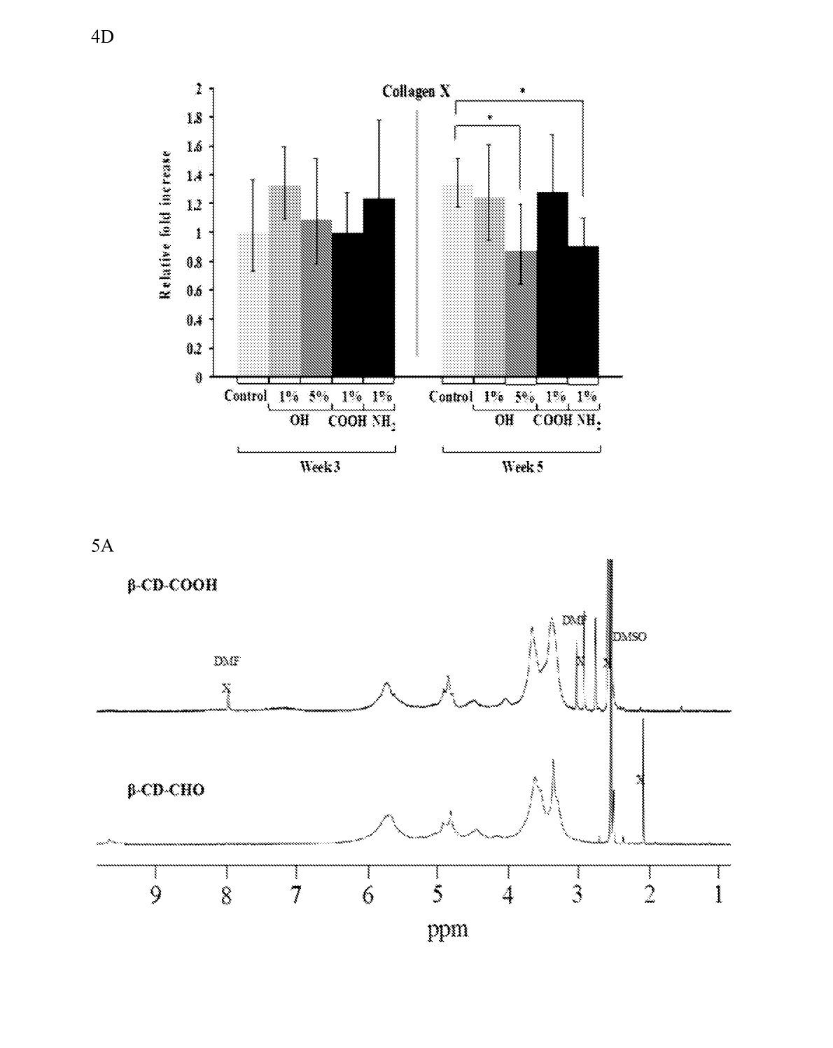

Synthesis of functionalized α-CDs. We employed simple synthetic schemes to synthesize α-CD with carboxylic acid groups and amine groups (Chem. Rev. 1998; 98:1977-96). For example, α-CDCOOH was synthesized in a two-step process. First, DMP was employed as a mild oxidizing agent to oxidize primary alcohols of α-CD into aldehydes (J Org Chem., 1983; 48:4155-6; Tetrahedron Lett., 1995; 36:8371-8374). This step resulted in randomly located aldehydes on the ring structure of α-CD (FIG. 1A). Second, its further oxidation by potassium peroxymonosulfate (Org. Lett., 2003; 5:1031-4) yielded a very water-soluble carboxylic acid functionalized α-CD (FIG. 1A). For synthesis of α-CD with amine groups, α-CDNH₂ was synthesized via N,N′-carbonyldiimidazole activation of OH groups (FIG. 1B). We preferred a monoamine-substituted α-CD to a multi-amine group containing α-CD due to its higher water solubility. ¹H-NMR, ¹³C-NMR and mass spectroscopy were performed to confirm the functionalization of CDs (FIGS. 1C & 5B). As shown in FIG. 1C, the new resonances on 1H-NMR spectrum of α-CDCHO and α-CDCOOH at ˜4.7 ppm and ˜5.5 ppm arose due to formation of the aldehyde groups (Tetrahedron Lett 1995; 36:8371-8374). Similarly, resonances at ˜3.2 ppm, ˜4.2 and 4.6 ppm appeared due to functionalization with ethylenediamine (FIG. 1C). ¹³C NMR performed on α-CDCOOH showed appearance of carbonyl (CO) at ˜170 ppm (FIG. 5B). MALDI-TOF spectrum modified α-CDs showed one to three hydroxyl groups were oxidized to aldehyde and carboxylic acid groups.

In addition, β-CDCHO and β-CDCOOH was synthesized using DMP and Oxone in a similar procedure to that of α-CDCHO and α-CDCOOH. As shown in FIG. 5A, a new resonance at ˜9.7 ppm on ¹H-NMR spectrum for aldehyde of β-CDCHO disappears for 3-CDCOOH, and a resonance at ˜170 ppm on ¹³C NMR spectrum appears for carboxylic acid of β-CDCOOH (FIG. 5B). MALDI-TOF spectra showed 1 to 3 hydroxyl groups of β-CDs were oxidized to aldehyde and carboxylic acid groups (FIG. 5A). The ease of synthesis of the randomly located carboxylic acid groups on β-CD makes it an ideal candidate for a water-soluble drug delivery carrier, eliminating the challenges of poor solubility of β-CD in aqueous solution.

### Example 2

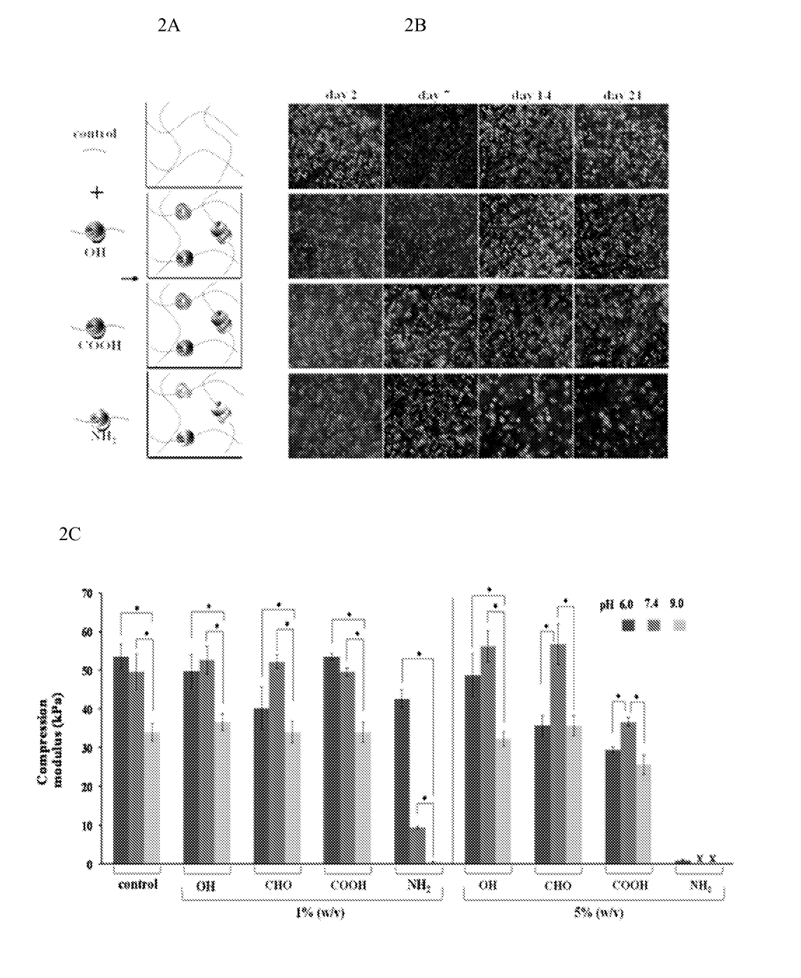

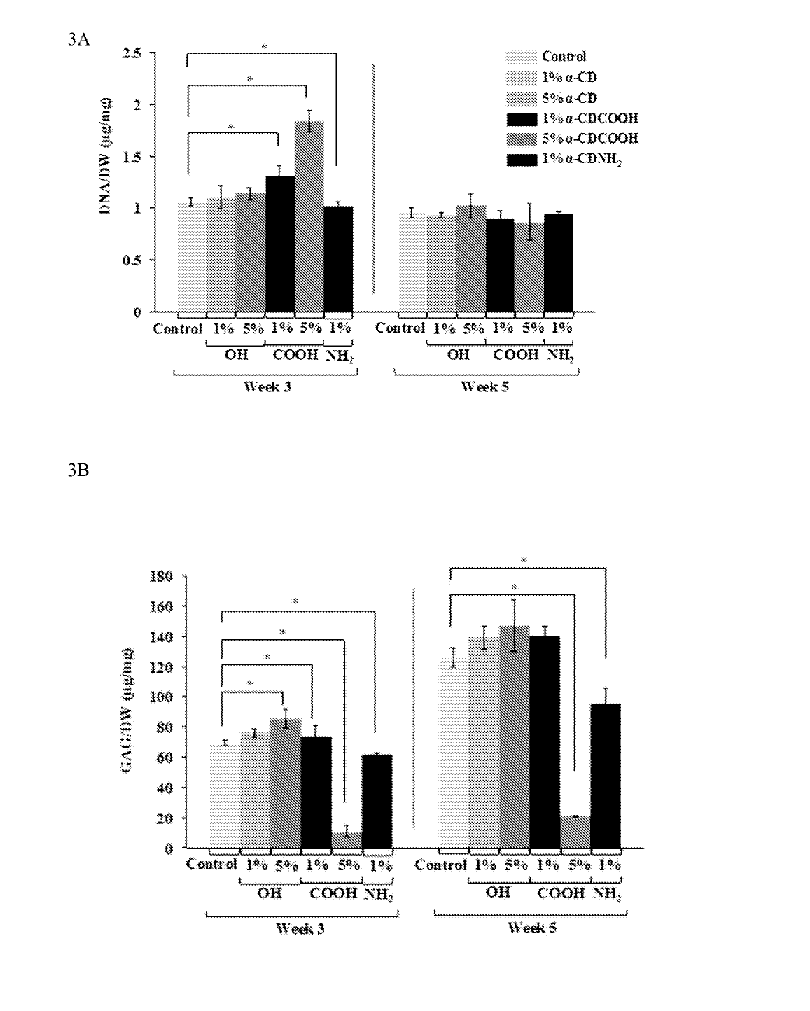

A cell viability study was performed on cell-encapsulated hydrogels containing functionalized α-CDs (1% and 5%, w/v) and PEGDA (10%, w/v) from day 2 to 3 weeks in chondrogenic medium. The live/dead staining on thin sections of cell-encapsulated hydrogels showed mostly viable cells (FIGS. 2A & 2B). On day 2, cells were uniformly distributed and mostly viable in all hydrogels. By day 14, cells started to cluster in both hydrogels with α-CD-OH and α-CDNH₂. However, amine-containing hydrogels showed formation of larger clusters and cells were more localized compared to those in other hydrogels.

### Example 3

Mechanical properties of the hydrogels. The compression modulus and swelling ratio of the hydrogels with PEGDA (10%, w/v) and various functionalized α-CDs (except amine) at both concentrations 1% and 5% (w/v) did not change significantly at a particular pH value (FIGS. 2C & 6). For amine-containing hydrogels, the pre-gel solution was made in an isotonic solution of pH=5.0. We believe that amine groups of α-CDNH₂ participate and compete with radical polymerization during photopolymerization (Prog Polym Sci 2006; 31:487-531). However, at higher pH, amines are deprotonated and are unavailable for any reactions with the vinyl bonds of PEGDA, which causes insufficient crosslinking of polymer chains. It is also evident from FIGS. 2C & 6 that by increasing the pH of the pre-gel solution, the compression modulus decreases and swelling ratio increases.

### Example 4

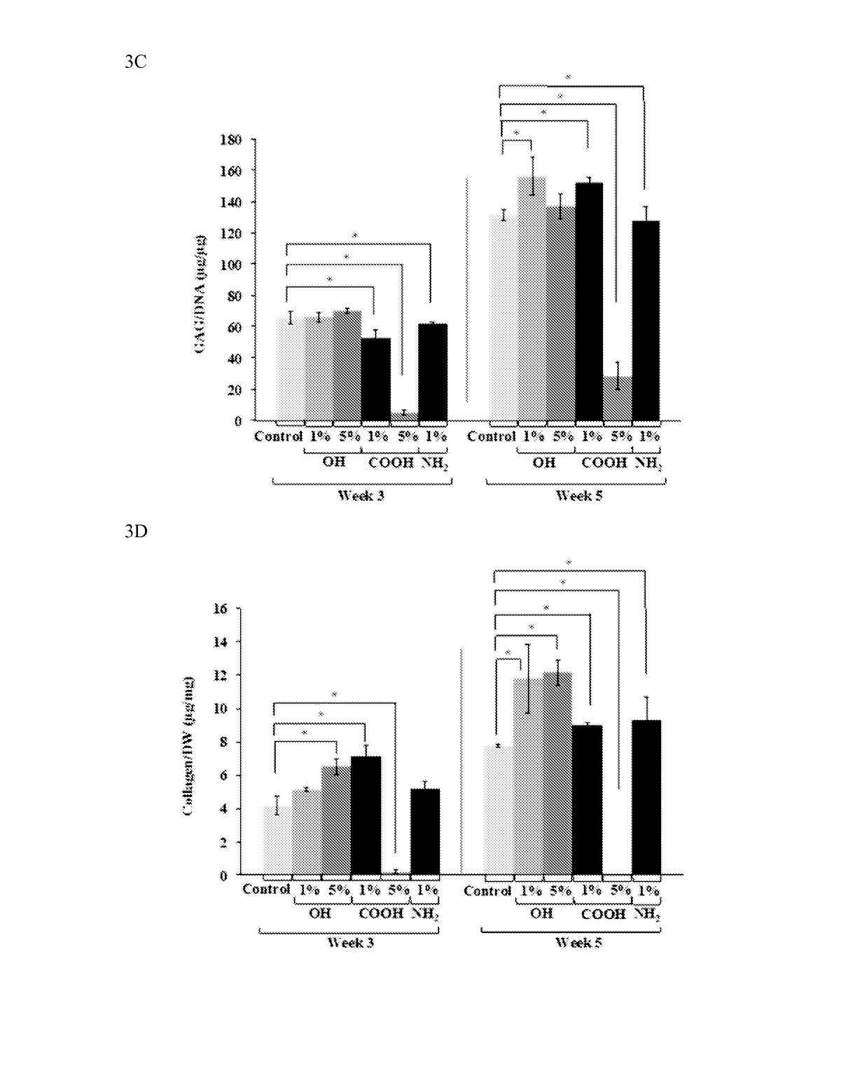

Chondrogenic differentiation of hMSCs in 3D PEG/α-CD hydrogels. Biochemical analysis was carried out to quantitatively illustrate the influence of functional α-CDs (1% and 5%, w/v, except amine) on chondrogenesis of hMSCs. As shown in FIG. 3A, the DNA/DW content values for the hydrogels with α-CDCOOH significantly increased after 3 weeks at both 1% and 5% (w/v) concentrations compared to the control, while it either remain the same or decreased slightly for hydrogels with α-CDOH and α-CDNH₂. However, after 5 weeks, no significant differences among these hydrogels were observed. GAG and collagen productions (normalized either to DW or DNA) in 5% (w/v) α-CDCOOH hydrogel samples were relatively negligible compared to control at both weeks 3 and 5 (FIG. 3B-3E). However, for hydrogels with 1% (w/v) α-CDCOOH, both GAG and collagen productions were either comparable or slightly higher than the control (FIG. 3B-3E). GAG and collagen productions were relatively unchanged by increasing the concentration of α-CDOH from 1% to 5% (w/v) in the hydrogels. However, by changing the concentration α-CDCOOH from 1% to 5% (w/v), both GAG and collagen productions were decreased by several orders. We also found that after 3 and 5 weeks in hydrogels with α-CDNH₂, GAG production was slightly lower than in the control. However, collagen production was similar to that of the control. This further supports our hypothesis that the chemical environment has significant influence on cell functions, specifically as a result of COOH groups.

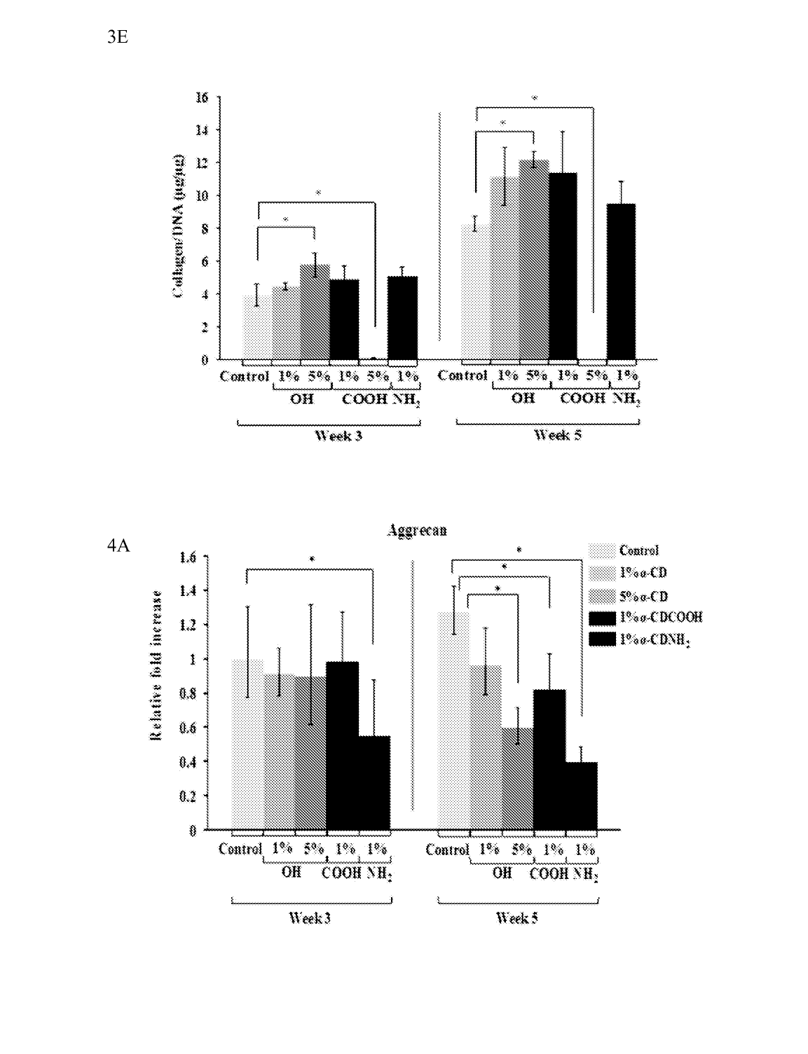

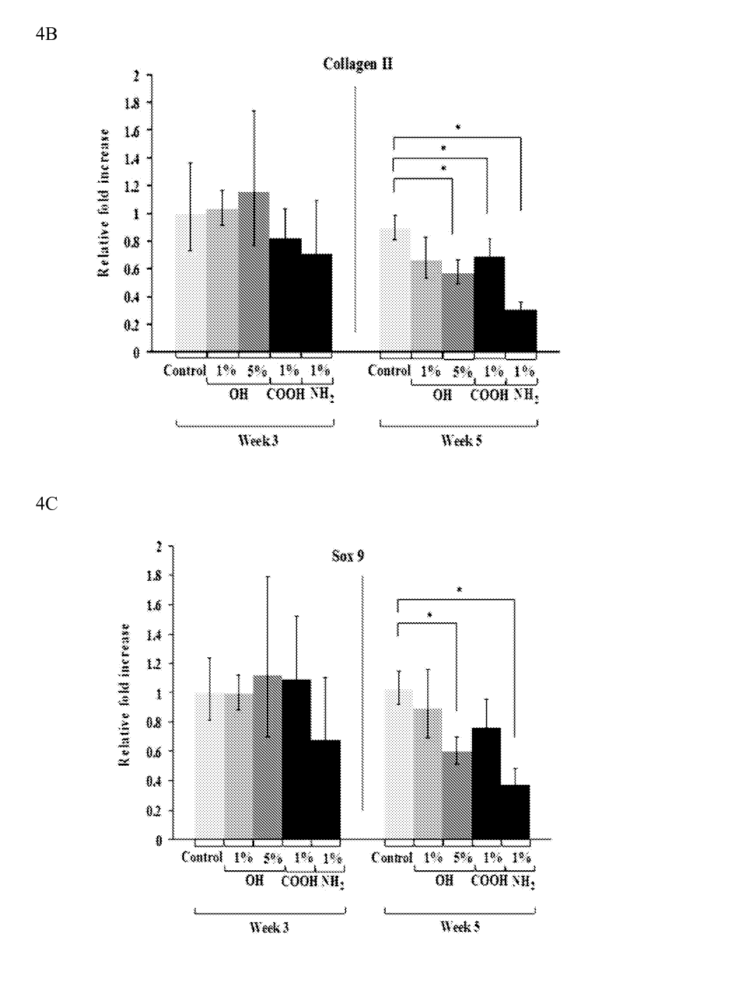

The relative gene-expression values for characteristic cartilage-specific markers were measured over time by PCR (FIG. 4A-4D). After 3 weeks of culturing cells in chondrogenic medium, the expressions of aggrecan, collagen II, sox9 and collagen X for functionalized α-CDs were similar to the control hydrogel. However, after 5 weeks, a decrease in relative expression values of these markers was observed. Specifically, cells in hydrogels with α-CDNH₂ expressed the lowest relative values.

Histological studies for cartilaginous ECM production were performed to follow the progression of chondrogenesis in functionalized α-CD (data not shown). Cellular morphology within the constructs was studied by H&E staining Significant morphological changes were noticed until 5 weeks in cultivation and, except for 5% (w/v) α-CDCOOH hydrogel samples, typical cartilage lacunae structures were obvious. By 3 weeks, positive safranin-O staining was observed for all the samples, except 5% (w/v) α-CDCOOH. After 5 weeks, more significant safranin-O staining diffused away from cells again, except for 5% (w/v) α-CDCOOH hydrogel. In constructs with 5% (w/v) α-CDNH₂, safranin-O staining was relatively less diffused and localized to cell clusters after both 3 and 5 weeks. Relatively stronger staining indicated maturation of neocartilage tissue at 5 weeks (data not shown).

### Example 5

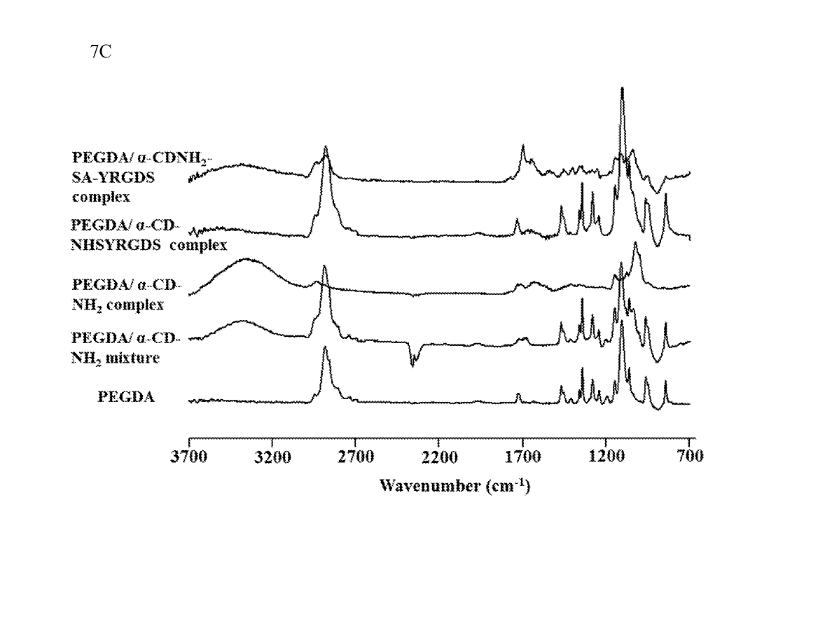

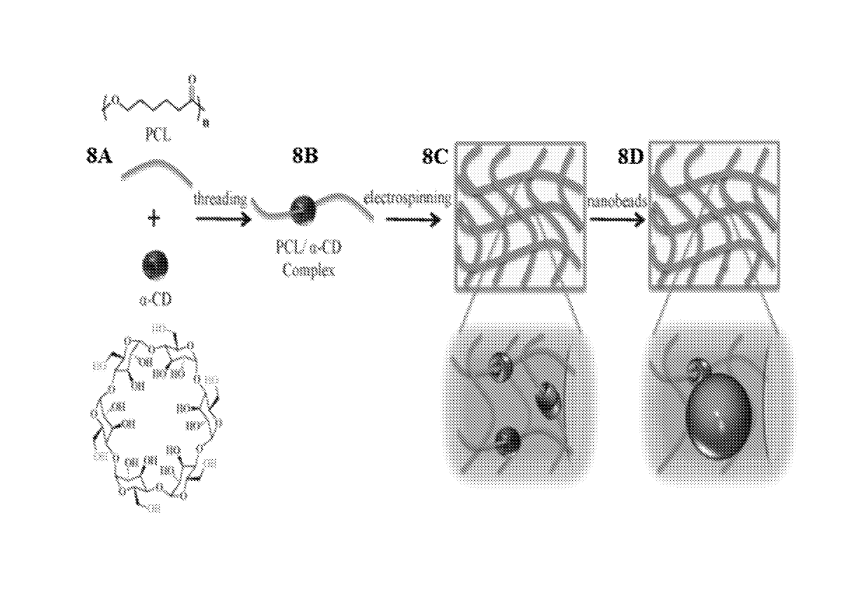

Characterization of PEG/α-CD hydrogels. Retention of threaded α-CD derivative in hydrogel was further analyzed by ninhydrin assay, XPS and FTIR-ATR spectroscopy. These hydrogels were washed several times with water and rigorously dried in a vacuum before the experiments Amines present in either α-CDNH₂ or YRGDS, or both formed a purple color complex with ninhydrin reagent (FIG. 7C) suggesting that YRGDS (SEQ ID NO: 17) were present in the hydrogels. FTIR-ATR spectra (FIG. 7D) of these dried hydrogels with threaded α-CDs were compared with control PEGDA hydrogel. A broader hydroxyl stretching peak at ˜3300 cm⁻¹ and multiple overlapped peaks at ˜1500-1700 cm⁻¹ corresponding to amide stretching confirmed the presence of α-CD and YRGDS in hydrogels. XPS analysis performed on these hydrogels also showed a peak at 400 eV that corresponds to nitrogen (FIG. 7E).

### Example 6

Applications of functionalized α-CD for creating cell-interactive hydrogels. The functionalized α-CD on PEG chains enabled us to conjugate biologically active moieties, such as an adhesion peptide (YRGDS (SEQ ID NO: 17)) (FIG. 7A). As an example, threaded α-CDNH₂ on PEGDA chains was conjugated to YRGDS by a bifunctional suberic acid-NHS linker (Sigma-Aldrich), while NHS modified α-CDCOOH was threaded onto PEGDA chains and conjugated with YRGDS (SEQ ID NO: 17) peptide. Cells adhered and spread on PEGDA/α-CDNH₂—YRGDS 2D hydrogel surface compared to PEGDA controls (FIG. 7B).

### Example 7

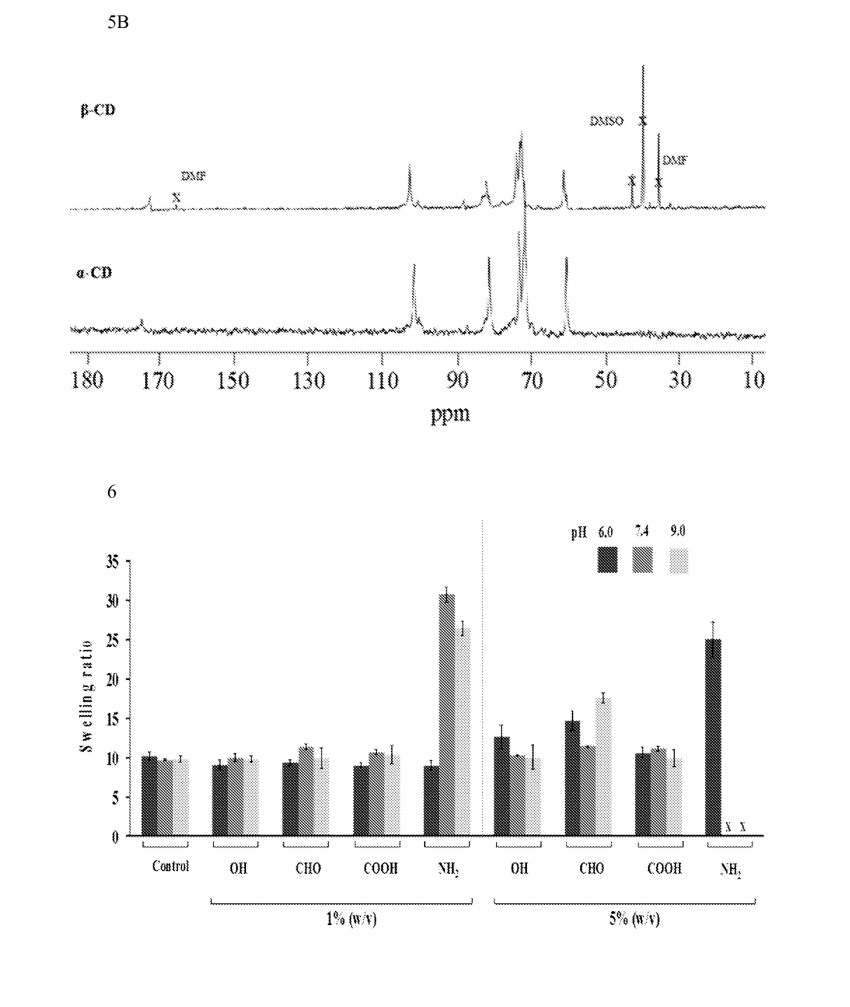

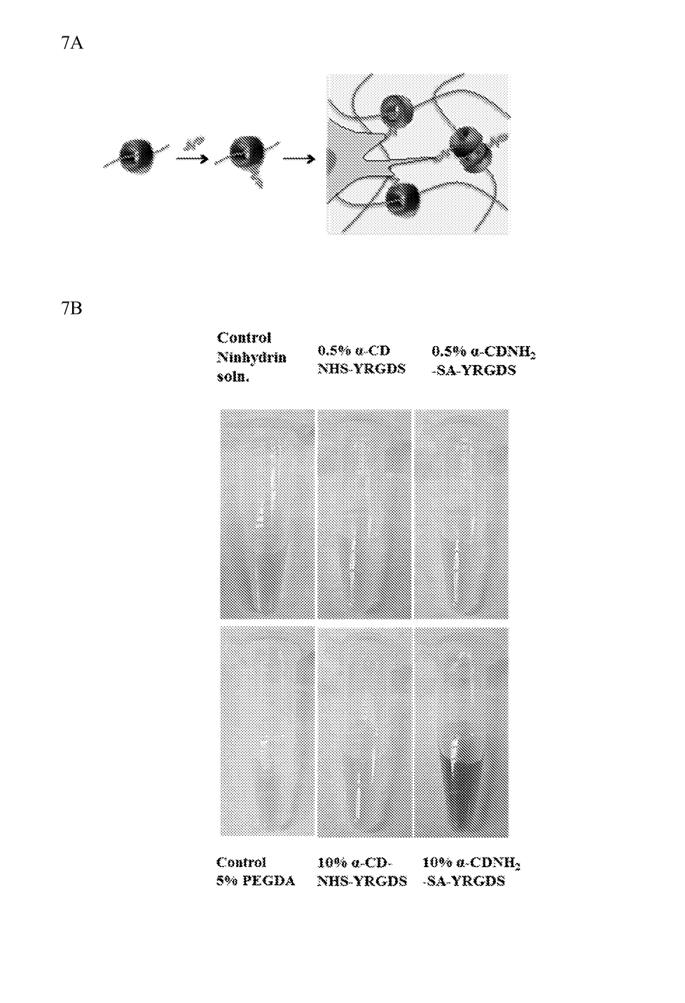

The present invention provides a PEG-based 3D hydrogel system to dictate cell functions by simply modulating material chemistry via decoration of the PEG chains with functionalized α-CDs. The PEG/functionalized α-CD-based hydrogel system of the present invention has unique features. First, PEG is chemically inert to cells and acts as an ideal polymer-platform for understanding the role of chemical functionalities when decorated with functionalized α-CDs. Second, unique chemical environments can be created by changing the type and amount of threaded α-CD molecules on PEG, while keeping the physical properties of the hydrogels unchanged. FIG. 2A shows that these hydrogels could support viability of hMSCs for a prolonged time, while keeping the mechanical properties of the hydrogels, e.g., compression modulus (FIG. 2B) and swelling ratio (FIG. 6) independent of the type and amount of functionalized α-CDs. Third, functionalized α-CDs on PEGDA chains can further be conjugated with biological components for creating more complex cell environment without chemically modifying the PEG main chain. An example of this is provided in an embodiment where a cell-adhesive peptide (Arg-Gly-Asp peptide sequence, or YRGDS (SEQ ID NO: 17)) conjugated α-CD was synthesized prior to its threading onto PEG chains. After threading, PEG chains were crosslinked to create a cell-responsive hydrogel. Functionalized α-CDs can be used to create cell-responsive hydrogels by first synthesizing and threading α-CDNH₂ and α-CDCOOH onto PEG chains followed by the attachment of a cell-adhesive peptide and crosslinking the PEG chains (FIGS. 7A-7E).

The material chemistry-dependent growth and chondrogenic differentiation of hMSCs was also investigated by encapsulating and culturing them in 3D hydrogels of PEG/functionalized α-CDs over 5 weeks. It was thought that chemical composition of the hydrogel can manipulate chondrogenic differentiation of hMSCs. Biochemical analysis performed for DNA/DW values after 3 weeks of culture in the chondrogenic medium showed cells proliferated significantly in hydrogels with α-CDCOOH, while the numbers of cells remain the same or slightly reduced in α-CDOH and α-CDNH₂ hydrogels, respectively (FIG. 3A). However, cells equally survived by 5 weeks, irrespective of the type of functionalized hydrogel (FIG. 3A). After 3 weeks, GAG and collagen productions in hydrogels with 1% α-CDOH were similar to that of the PEG hydrogels; however, after 5 weeks these values significantly increased compared to that of PEG control. An increasing trend of collagen (normalized to both DNA and DW) and GAG (normalized to DW) productions was also observed with increasing concentrations of α-CDOH from 1% to 5% (w/v) (FIGS. 3B-3D), irrespective of time period. GAG and collagen produced in hydrogels with 1% (w/v) α-CDCOOH were comparable to the control; however, these values were many times lower for 5% (w/v) α-CDCOOH hydrogels. Cells in 5% (w/v) α-CDCOOH hydrogels produced minimal GAG and collagen (FIGS. 3B-3E). After 3 and 5 weeks, GAG production in hydrogels with 1% α-CDNH₂ was slightly lower compared to that of the control, while collagen production remained similar to that of control. All hydrogels, except 5% α-CDCOOH, could increase GAG and collagen productions with time. These results show that all these functionalities at lower concentrations support chondrogenesis to a similar extent; however, at higher concentrations, α-CDOH promotes and α-CDCOOH suppresses chondrogenesis of hMSCs. PCR studies showed that after 3 weeks, the relative gene expressions for chondrogenic markers, except aggrecan were similar for all hydrogels (FIGS. 4A-4D). After 5 weeks of culture, the relative gene expression values were slightly lower in all α-CD decorated hydrogels compared to PEGDA control (FIGS. 4A-4D). To account for this observation of slower chondrogenesis in PEGDA samples compared to hydrogels with α-CDs, it is thought that these gene markers might have saturated at early time points in α-CD decorated hydrogels and reached a point where these are not upregulated anymore, as tissue formation occurred. Histological studies for cellular morphology and cartilaginous ECM production showed typical cartilage lacunae structures and GAG production in all hydrogel samples, except for 5% (w/v) α-CDCOOH hydrogel (data not shown). Similar to biochemical analysis, histology supported the observation that the lower concentration of α-CDCOOH promoted chondrogenesis, while higher concentration suppressed tissue formation. The findings show that lineage-specific stem cell differentiation and tissue formation can be directed by controlling matrix chemistry via the type and amount of chemical functionalities in the hydrogels of the present invention.

### Example 8

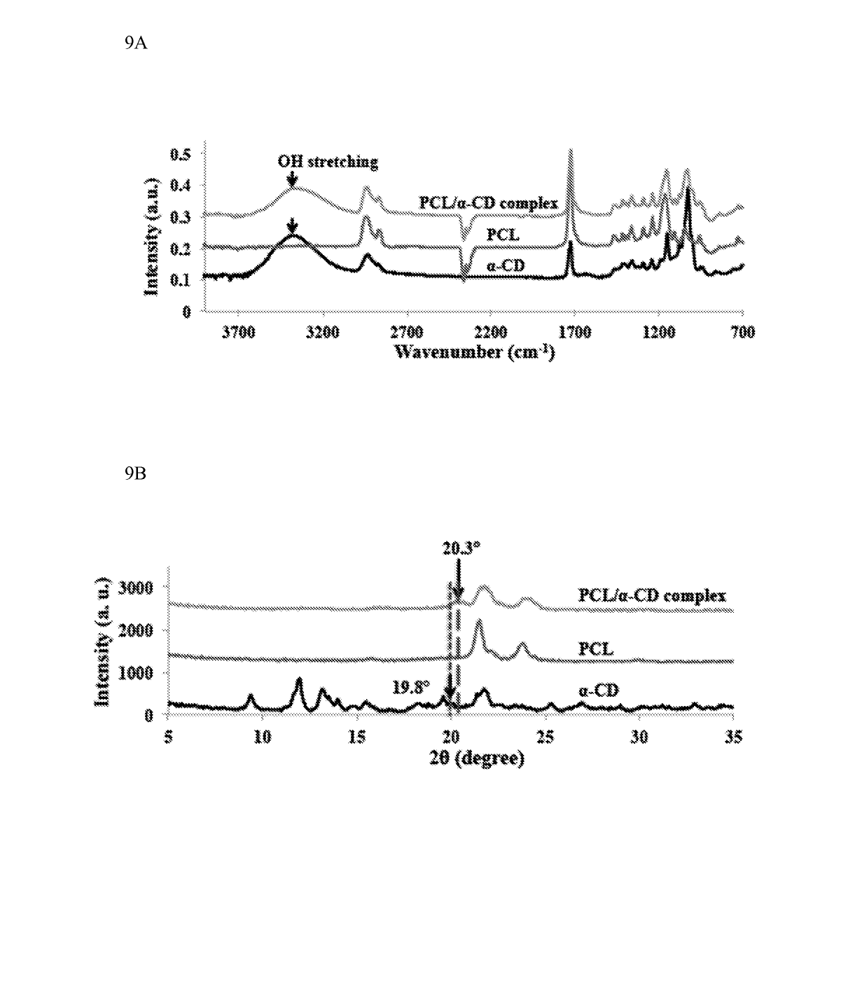

Multifunctional electrospun nanofibers of the present invention were developed based on the inclusion complex (IC) of aliphatic polyester-α-cyclodextrin (e.g., PCL-α-CD) for tissue engineering applications (FIGS. 8A-8D). α-CD is a six-member oligosaccharide doughnut ring structure with an inner cavity (diameter ˜0.6 nm) and an outside diameter of ˜1.4 nm. 34 α-CD rings physically thread onto the PCL chains via non-covalent interactions and resemble a molecular necklace structure (FIGS. 8A-8B). α-CD bears hydroxyl groups that can be modified to create a variety of functionalities that also allow conjugation of multiple bioactive agents or ligands.

PCL-α-CD IC was synthesized (FIGS. 8A-8B), and then was electrospun into nanofibers (FIG. 8C). The utility of functional groups on the nanofibers was demonstrated by conjugating a polymeric nanobead (FIG. 8D) and using the electrospun fiber as a scaffold for in vitro stem cell culture and differentiation for bone tissue formation, based on the inclusion complex (IC) of aliphatic polyester-α-cyclodextrin (e.g., PCL-α-CD) for tissue engineering applications (FIGS. 8A-8D). α-CD is a six-member oligosaccharide doughnut ring structure with an inner cavity (diameter ˜0.6 nm) and an outside diameter of ˜1.4 nm. α-CD rings physically thread onto the PCL chains via non-covalent interactions and resemble a molecular necklace structure (FIGS. 8A-8B). α-CD bears hydroxyl groups that can be modified to create a variety of functionalities that also allow conjugation of multiple bioactive agents or ligands.

### Example 9

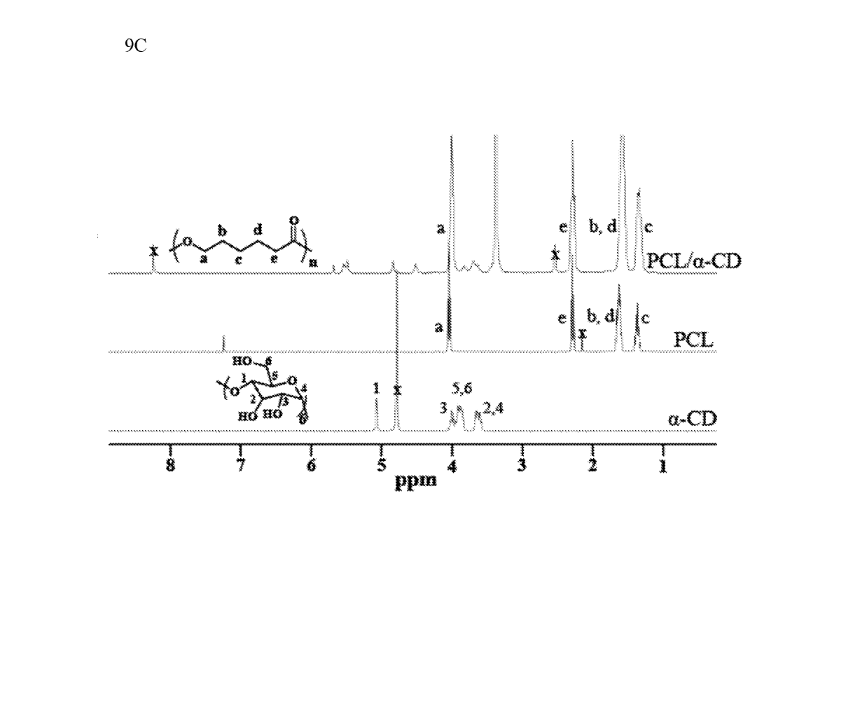

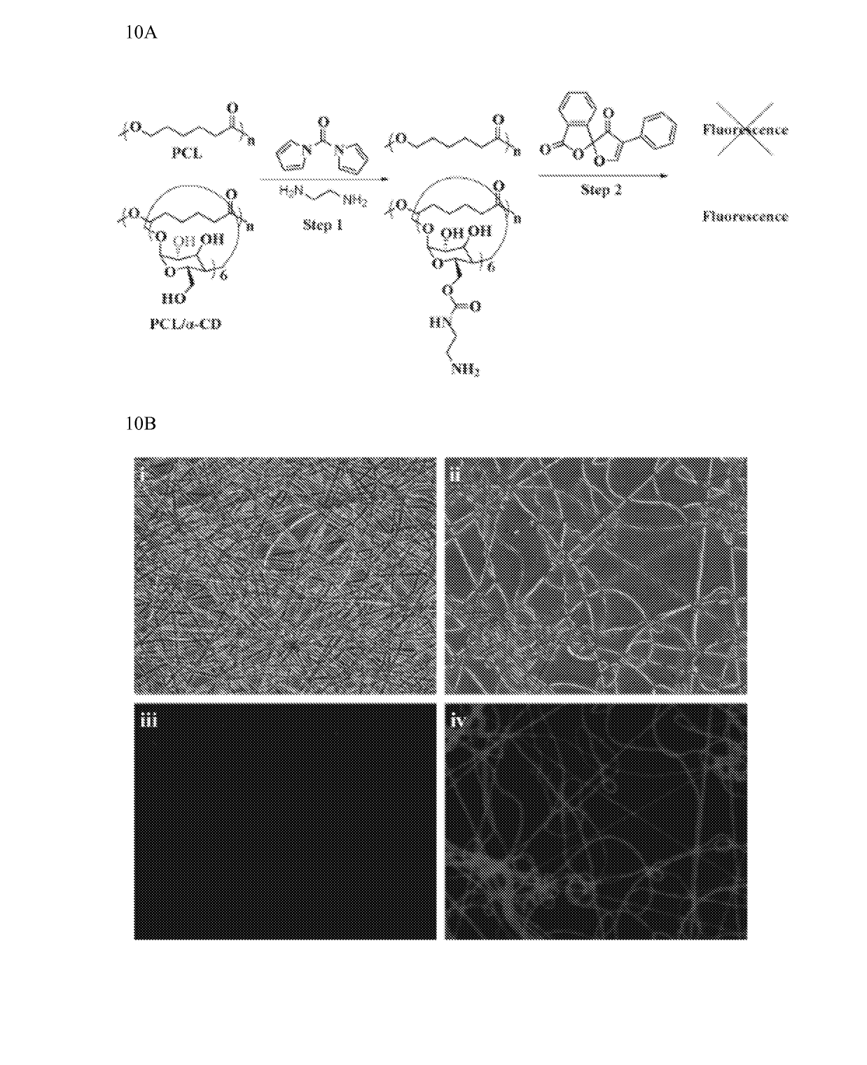

Material characterization of PCL α-CD. PCL-α-CD IC was characterized for threading of α-CD on PCL chains by FTIR-ATR, WAXD and ¹H NMR spectroscopy. FTIR-ATR screening of PCL-α-CD IC, PCL and α-CD showed three peaks at 1026 cm-1, 1079 cm-1 and 1158 cm-1 and confirmed the presence of α-CD. A distinct stretching band at 1735 cm-1 appeared as a result of the carbonyl bonds of PCL (FIG. 9A). A broad band at 3382 cm-1 appeared because of the symmetric and antisymmetric OH stretching of α-CD in PCL-α-CD IC, which is absent in PCL. Also, in contrast to the α-CD spectrum, a slight shift of the OH stretching band in the IC arose resulting from the formation of hydrogen bonds between α-CD and its guest polymer in the channel form.

### Example 10

WAXD result showed that PCL exhibited two typical strong peak reflections at 20=22° and 23.8°, while α-CD displayed a series of peaks at 9.9°, 12.2°, 14.5°, 19.8° and 21.9° as previously reported (FIG. 9B). In PCL-α-CD IC, most crystalline diffraction peaks due to PCL disappeared, which indicated suppression of guest crystallization by formation of IC. New peaks at ˜20° and ˜22.5° appeared due to formation of IC. The molar ratio of the two components in PCL-α-CD IC was quantified by integration of resonances for the ¹H NMR spectra of α-CD and PCL (shown in FIG. 9C).

### Example 11

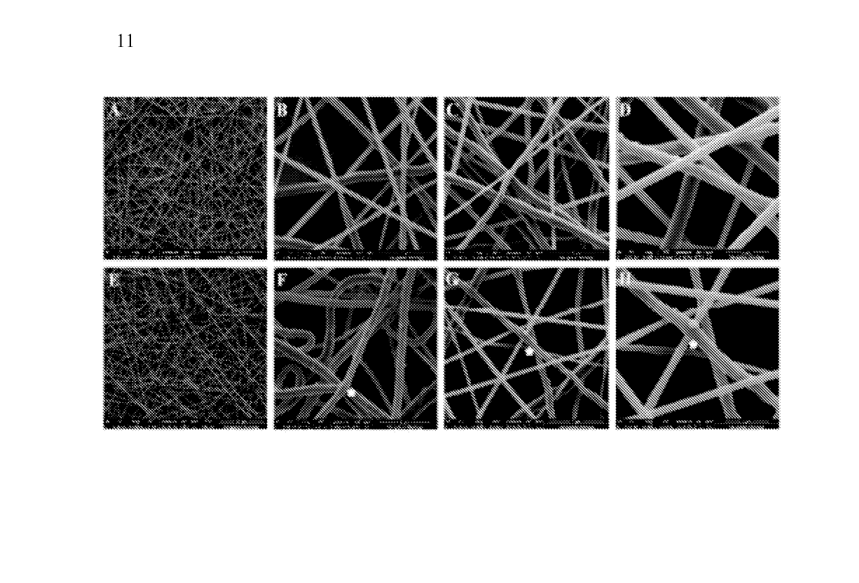

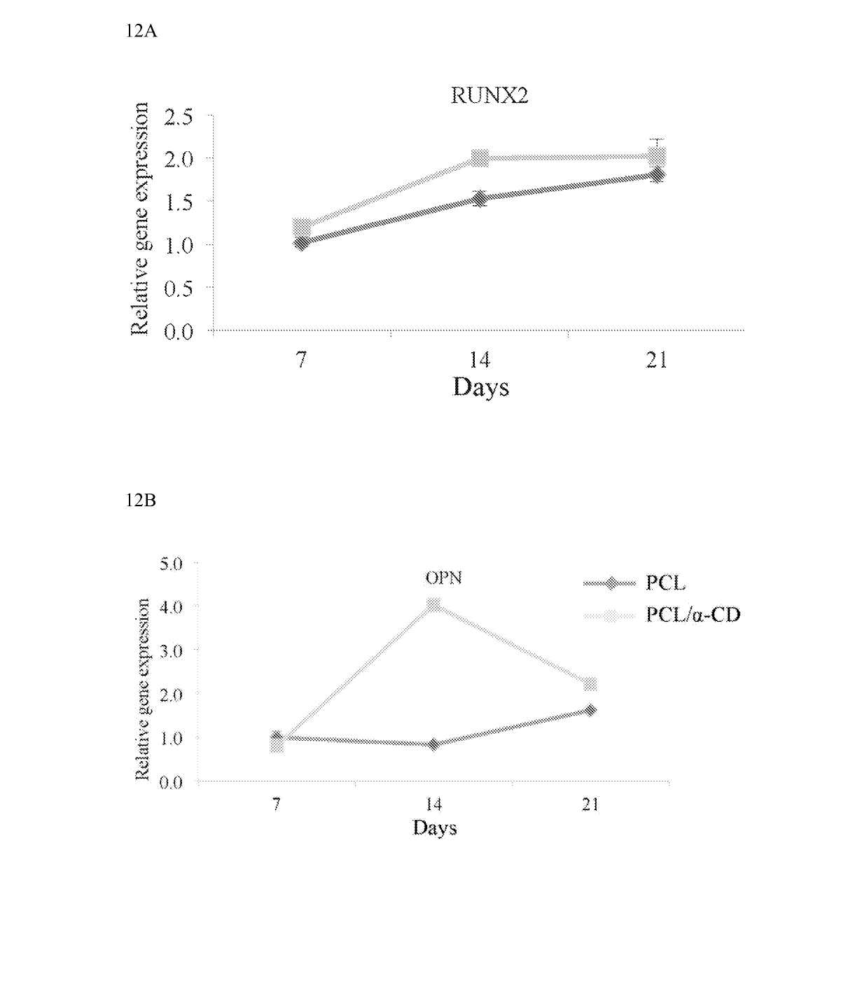

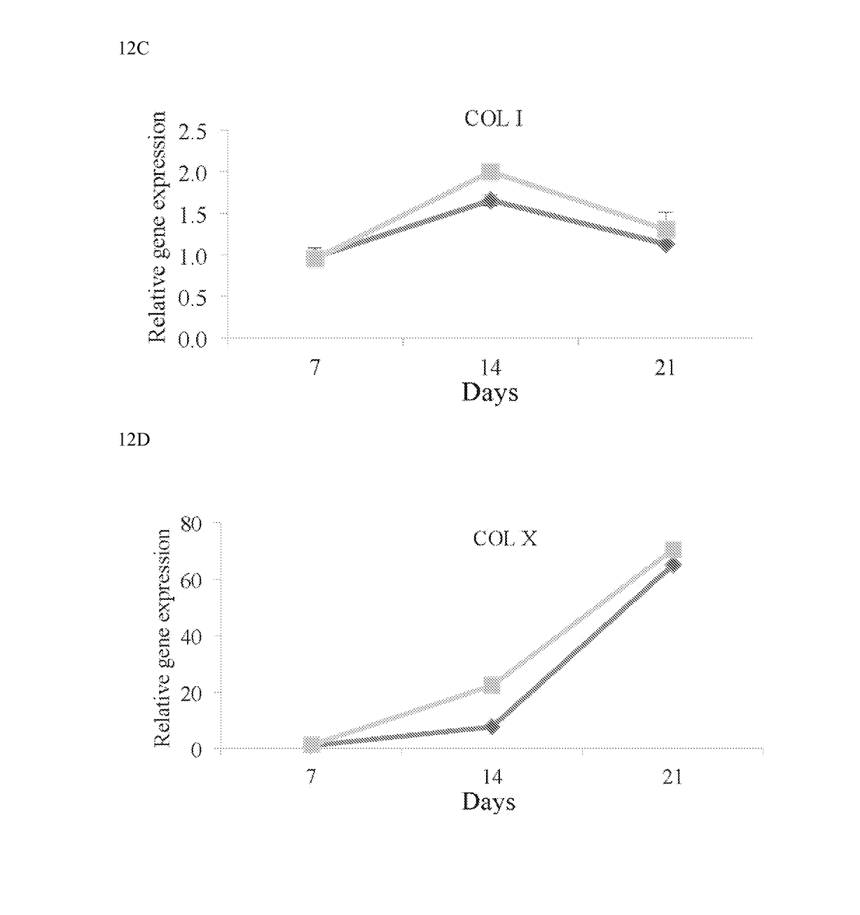

Nanofiber synthesis, characterization and modification PCL α-CD. Unlike PCL alone, neither DMSO nor CH₂Cl₂ dissolved IC completely and, therefore, was unsuitable for electrospinning of the polymer solution. However, the polymer was successfully dissolved and electrospun in a mixture of DMSO/CH₂Cl₂ (3/2, v/v). These fibers were also tested for retention of threaded α-CD on PCL chains by utilizing the hydroxyl groups of α-CD on the surface for further chemical modifications and conjugations. First, both PCL and PCL-α-CD fibers were activated by CDI (FIG. 10A); second, CDI-activated hydroxyl groups were modified to amine groups by reacting with a short length diamine (e.g., ethylenediamine) (FIG. 10A). Subsequently, an amine-reactive fluorescent molecule, fluorescamine, was conjugated onto the fiber surfaces (FIG. 10A). The fibers modified with fluorescamine turned blue under UV light exposure (FIG. 10B). The hydroxyl groups on the nanofibers were also utilized to conjugate a structural component (amine-containing polystyrene nanobeads). SEM images at higher magnification showed no conjugated nanobeads on PCL fibers (FIGS. 11A-11D); however, PCL-α-CD fibers were decorated with nanobeads via hydroxyl sites (FIGS. 11E-11H). The CDI-untreated PCL or PCL-α-CD fibers did not conjugate to nanobeads.

### Example 12

Cell response to PCL-α-CD nanofibers PCL α-CD. hADSCs' viability and spreading were studied over 3, 7, 14 and 21 days with LIVE/DEAD® (Invitrogen™, Life Technologies) and F-actin staining hADSCs attached to both PCL and PCL-α-CD fibers, and exhibited an elongated fibroblast-like morphology after 3 days, which indicated a viable state (data not shown). A continuous increase in cell number was visually observed for both fibers. After 21 days, LIVE/DEAD® staining determined the presence of 96.5±2.5% and 97.1±1.5% live cells for PCL and PCL-α-CD fibers, respectively.

Positive staining with alizarin red and ALP was observed on both fibers, which confirmed calcium deposition and mineralization. A substantial increase in the intensity of alizarin red staining was observed from days 14 to day 21 on both fibers, suggesting that by day 21, mineral deposition was greatly enhanced (data not shown).

### Example 13

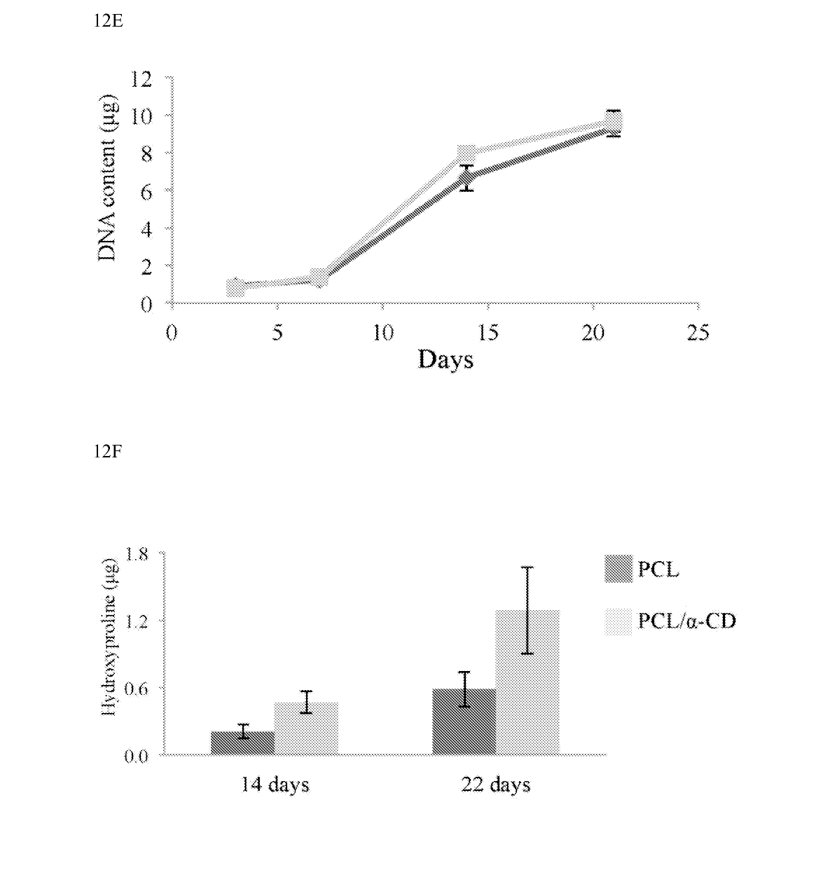

Quantitative analysis of osteogenic gene-expression. Four osteogenic markers were selected for this study: osteogenesis transcription factor Runx2, and three bone collagen structural proteins: osteopontin, collagen type I and collagen type X. In general, PCL-α-CD fibers induced greater amounts of osteogenic gene expression compared to PCL fibers (FIGS. 12A-12D). Similarly, relatively higher collagen deposition was obtained on PCL-α-CD fibers (FIGS. 12E-12F). In summary, ADSCs proliferated at a similar rate on both types of fibers, while PCL-α-CD fibers enhanced osteogenesis.

### Example 14

The PCL-α-CD-based electrospun nanofibrous scaffold of the present invention has unique advantages: first, it is as easy to fabricate as PCL fibers; second, it has multiple functional sites for further conjugation and third, it is independent of the PCL-main chain modification as α-CD physically threads onto PCL chains. The ease of conjugation of various chemical and biological components to create user-specific unique cell environments without PCL modification, makes these nanofibers a powerful biomaterial tool for tissue engineering. For example, the utility of the hydroxyl groups of the α-CD on the fiber surface is illustrated by the conjugation of a fluorescent small molecule, fluorescamine, and a polystyrene nanobead (FIGS. 10 & 11). Similarly, various small molecules; cell-interactive peptides, such as the cell-binding peptide Arg-Gly-Asp (RGD) and other biological components can also be conjugated to improve cell-binding capability of the nanofibers and provide necessary chemical and biological signals for cell functions. In an alternate embodiment, cell adhesion can be improved on PCL nanofibers by co-electrospinning PCL with naturally derived materials, including gelatin or mineralized ECM. In a further embodiment the PCL-α-CD nanofibers of the present invention can be used for the controlled release of biological components from the fiber surface. Bioactive components can be conjugated to the PCL-α-CD nanofibers of the present invention via external-stimulus-sensitive bonds through functionalized CDs, such as hydrolyzable ester or photocleavable bonds. This allows the bioactive components to have a greater sustained-release time profile, which is highly desirable in a scaffold design for controlled drug release.

### Example 15

Application of PCL-α-CD nanofibers as a 2D substrate for cell growth and osteogenic differentiation potential of hADSCs was investigated. Recently, there has been much attention focused on hADSCs because of their biological similarity to hBM- (human bone marrow-) MSCs, ease of isolation through abundant and readily accessible adipose tissue, replication capability and multi-lineage differentiation potential. This makes hADSCs invaluable sources of adult stem cells for bone tissue engineering applications. It was thought that PCL-α-CD nanofibers can be employed as a scaffold for osteogenic differentiation of hADSCs, as can PCL nanofibers. Therefore, hADSCs were cultured onto 2D substrates of PCL and PCL-α-CD nanofibers in osteogenic media. Morphologically, cells were fully extended and elongated at early time points, indicating cell viability and adhesion (data not shown). By three weeks, hADSCs appeared to be completely integrated into the structure of the fibers; however, cells on PCL-α-CD fibers appeared more aligned than on PCL fibers.

The extent of osteogenic differentiation of hADSCs on nanofibers was monitored by gross-images of positive staining for calcium mineralization and alkaline phosphatase (ALP) activity (data not shown). ALP is an enzyme responsible for dephosphorylation of phosphates and initiating mineralization of ECM, which induces matrix mineralization by restricting matrix nucleation inhibitors. However, ECM mineralization occurs in the later stage of osteogenic differentiation and requires long-term culture before any measurable matrix production occurs. As such, ALP is regarded as an early-stage marker in osteogenesis, and its turning to plateau from up-regulation is considered a signal for the initiation of mineralization. A substantial increase in the intensity of alizarin red staining was observed from day 14 to day 21 on both fibers, suggesting that by day 21, mineral deposition was greatly enhanced (data not shown). However, ALP staining did not show much visual difference between PCL and PCL-α-CD samples. This might be due to a possible plateauing of ALP generation at the mid-to-later stage of osteogenesis.

### Example 16

PCR studies showed that ADSCs seeded onto PCL-α-CD nanofibers of the present invention exhibited equal or marginally higher relative expressions of osteogenesis markers than on PCL fibers, as shown in FIGS. 12A-12D. The selected markers are critical transcription factors or proteins involved in osteogenesis. Runx2 is an important transcription factor during osteogenesis, while osteopontin, collagen type I and collagen type X are main structural proteins of collagens found in bone. As we observed in this study, their higher expressions with time indicated a greater tendency to differentiate into bone-related cell types. We also found that while the DNA content of the two samples remained the same at each time point (FIG. 12E), collagen deposition on PCL-α-CD fibers was significantly higher than on PCL fibers (FIG. 12F). An increase in DNA content over time indicated that cells proliferated equally well on both PCL-α-CD and PCL fibers (FIG. 12E). Collagen is a major organic component of mineralized ECM, comprising ˜90% of all the organic material in bone, and it serves as a template for mineral deposition. Our result suggests that the PCL-α-CD fibers could enhance collagen production, making a relatively better substrate to induce bone formation. Furthermore, the chemical composition of the nanofiber with α-CD played an important role in cell growth and differentiation, while fiber morphology or topography was unchanged. Earlier, we showed that functionalized α-CDs in PEG hydrogels could enhance tissue formation. These findings support the concept that α-CDs promote stem cell differentiation into musculoskeletal tissues, regardless of the type of polymers used for creating an artificial environment in the form of hydrogels or nanofibrous scaffolds.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A multifunctional biomaterial comprising:
one or more biocompatible polymers and one or more α-cyclodextrin molecules having a plurality of hydroxyl groups capable of being chemically substituted with another functional group or moiety;
wherein the one or more biocompatible polymers have at least 10 or more monomeric units; and
wherein the one or more biocompatible polymers are included in the cavities of the one or more α-cyclodextrin molecules in a skewered manner to obtain a pseudopolyrotaxane configuration.

2. The multifunctional biomaterial of claim 1, wherein the biocompatible polymer is a block copolymer.

3. The multifunctional biomaterial of claim 2, wherein the biocompatible polymer is hydrophilic.

4. The multifunctional biomaterial of claim 3, wherein the hydroxyl groups of the one or more α-cyclodextrin molecules are chemically substituted with another functional group or moiety selected from the group consisting of hydrophobic groups, hydrophilic groups, peptides, C₁-C₆ alkyl, C₂-C₆ alkenyl, C₂-C₆ alkynyl, C₁-C₆ hydroxyalkyl, C₁-C₆ alkoxy, C₁-C₆ alkoxy C₁-C₆ alkyl, C₁-C₆ alkylamino, di-C₁-C₆ alkylamino, C₁-C₆ dialkylamino C₁-C₆ alkyl, C₁-C₆ thioalkyl, C₂-C₆ thioalkenyl, C₂-C₆ thioalkynyl, C₆-C₂₂ aryloxy, C₂-C₆ acyloxy, C₂-C₆ thioacyl, C₁-C₆ amido, C₁-C₆ sulphonamido, C₁-C₆ carboxyl and derivatives, phosphonates and sulfones.

5. The multifunctional biomaterial of claim 4, wherein the biocompatible polymer is selected from the group consisting of: Poly(ethylene glycol), Poly(propylene glycol), Poly(methyl vinyl ether), Oligoethylene, Poly(isobutylene) Poly(tetrahydrofuran) Poly(oxytrimethylene), Poly(dimethylsiloxsane), Poly(dimethylsilane), Nylon 6, Nylon 11, Poly(acrylonitrile), Squalane, Poly(1,3-dioxolane), Poly(iminooligomethylene), Poly(l-lysine), Polyethyleneimine, Poly(adipate), Poly(l-caprolactone), Poly(L-lactic acid), or derivatives thereof.

6. The multifunctional biomaterial of claim 5, wherein the one or more biocompatible polymers are mono, or disubstituted with an acrylate group.

7. The multifunctional biomaterial of claim 6, wherein the one or more biocompatible polymers is poly(ethylene glycol) diacrylate (PEGDA).

8. The multifunctional biomaterial of claim 5, wherein the biocompatible polymer is hydrophobic.

9. The multifunctional biomaterial of claim 5, wherein the biocompatible polymer is polycaprolactone, or a derivative thereof.

10. The multifunctional biomaterial of claim 1, wherein the one or more α-cyclodextrin molecules have their hydroxyl groups substituted with one or more integrin binding peptides.

11. The multifunctional biomaterial of claim 10, wherein the integrin binding peptide is YRGDS (SEQ ID NO: 17).

12. The multifunctional biomaterial of claim 1, wherein the one or more α-cyclodextrin molecules have their hydroxyl groups substituted with an aldehyde, a carboxylic acid group, or an amino group.

13. The multifunctional biomaterial of claim 1, wherein the biomaterial is 2-dimensional.

14. The multifunctional biomaterial of claim 1, wherein the biomaterial is 3-dimensional.

15. The multifunctional biomaterial of claim 5, wherein the biocompatible polymer is PEG and the biomaterial is in the form of a hydrogel.

16. The multifunctional biomaterial of claim 5, wherein the biocompatible polymer is PCL and the biomaterial is in the form of a nanofiber.

17. A hydrogel biomaterial comprising one or more poly(ethylene glycol) polymers and one or more α-cyclodextrin molecules having a plurality of hydroxyl groups capable of being chemically substituted with another functional group or moiety selected from the group consisting of hydrophobic groups, hydrophilic groups, peptides, C₁-C₆ alkyl, C₂-C₆ alkenyl, C₂-C₆ alkynyl, C₁-C₆ hydroxyalkyl, C₁-C₆ alkoxy, C₁-C₆ alkoxy C₁-C₆ alkyl, C₁-C₆ alkylamino, di-C₁-C₆ alkylamino, C₁-C₆ dialkylamino C₁-C₆ alkyl, C₁-C₆ thioalkyl, C₂-C₆ thioalkenyl, C₂-C₆ thioalkynyl, C₆-C₂₂ aryloxy, C₂-C₆ acyloxy, C₂-C₆ thioacyl, C₁-C₆ amido, C₁-C₆ sulphonamido, C₁-C₆ carboxyl and derivatives, phosphonates and sulfones.
wherein the one or more poly(ethylene glycol) polymers have at least 10 or more monomeric units; and
wherein the one or more poly(ethylene glycol) polymers are included in the cavities of the one or more α-cyclodextrin molecules in a skewered manner to obtain a pseudopolyrotaxane configuration.

18. The hydrogel biomaterial of claim 17, wherein the hydrogel is cross-linked via the terminal ends of the polymer chains.

19. A method for making a hydrogel biomaterial comprising:
a) obtaining a solution of α-cyclodextrin molecules in a suitable biologically compatible aqueous buffer;
b) adding to a) a sufficient amount of hydrophilic polymers or derivatives thereof in a suitable biologically compatible aqueous buffer to create a solution having a polymer concentration of about 1 to about 20% (w/v) and a α-cyclodextrin concentration of about 0.1 to about 10% (w/v);
c) mixing the solution of b) for a sufficient time to provide an inclusion step in which hydrophilic polymers or derivatives thereof and cyclodextrin molecules obtain a pseudopolyrotaxane configuration in which the hydrophilic polymers or derivatives thereof are included in the cavity of each of α-cyclodextrin molecule in a skewered manner;
d) adding a photoinitiator to the solution of c) to create a final concentration of photoinitiator of between about 0.01 to about 0.1% (w/v);
e) exposing the solution of d) to electromagnetic radiation at a wavelength specific to the photoinitiator for a sufficient amount of time to initiate the polymerization of the polymers in the solution; and
f) allowing the polymerization to complete.

20. A method for making a 2-dimensional cell-encapsulated hydrogel comprising:
a) obtaining a solution of α-cyclodextrin molecules in a suitable biologically compatible aqueous buffer and placing it in a shallow dish or container or similar support;
b) adding to a) a sufficient amount of hydrophilic polymers or derivatives thereof in a suitable biologically compatible aqueous buffer to create a solution having a hydrophilic polymer concentration of about 1 to about 20% (w/v) and a α-cyclodextrin concentration of about 0.1 to about 10% (w/v);
c) mixing the solution of b) for a sufficient time to provide an inclusion step in which hydrophilic polymers or derivatives thereof and α-cyclodextrin molecules obtain a pseudopolyrotaxane configuration in which the hydrophilic polymers or derivatives thereof are included in the cavity of each of α-cyclodextrin molecule in a skewered manner;
d) adding a photoinitiator to the solution of c) to create a final concentration of photoinitiator of between about 0.01 to about 0.1% (w/v);
e) exposing the solution of d) to electromagnetic radiation at a wavelength specific to the photoinitiator for a sufficient amount of time to initiate the polymerization of the polymers in the solution;
f) soaking the polymerized gel of e) for a sufficient period of time to remove any α-cyclodextrin which do not have the hydrophilic polymers or derivatives thereof are included in their cavities; and
g) seeding a quantity of cells onto the polymerized gel of f) at a density of between about 5000 to about 50,000 cells/cm² in a biologically compatible growth media.

21. A method for making a 3-dimensional cell-encapsulated hydrogel comprising:
a) obtaining a solution of α-cyclodextrin molecules in a suitable biologically compatible aqueous buffer and placing it in a container or similar support;
b) adding to a) a sufficient amount of hydrophilic polymers or derivatives thereof in a suitable biologically compatible aqueous buffer to create a solution having a hydrophilic polymer concentration of about 1 to about 20% (w/v) and a α-cyclodextrin concentration of about 0.1 to about 10% (w/v);
c) mixing the solution of b) for a sufficient time to provide an inclusion step in which hydrophilic polymers or derivatives thereof and α-cyclodextrin molecules obtain a pseudopolyrotaxane configuration in which the hydrophilic polymers or derivatives thereof are included in the cavity of each of α-cyclodextrin molecule in a skewered manner;
d) adding a photoinitiator to the solution of c) to create a final concentration of photoinitiator of between about 0.01 to about 0.1% (w/v);
e) seeding a quantity of cells into the solution of d) at a quantity of between about 500,000 to about 5×10⁶ cells in a biologically compatible growth media; and
f) exposing the solution of e) to electromagnetic radiation at a wavelength specific to the photoinitiator for a sufficient amount of time to initiate the polymerization of the polymers in the solution.

22. The method of claim 21, wherein the cells are mammalian cells.

23. The method of claim 22, wherein the mammalian cells are mesenchymal stem cells, cardiac stem cells, liver stem cells, retinal stem cells, and epidermal stem cells.

24. A method for making a multifunctional biomaterial comprising:
a) obtaining a sufficient amount of hydrophobic biocompatible polymers or derivatives thereof in a suitable organic solvent to create a solution having a polymer concentration of about 0.1 to about 0.2 g/mL polymer and heating the solution to about 45° C. to 60° C.;
b) adding to a) a solution of α-cyclodextrin molecules in a suitable polar aprotic solvent at a concentration of about 0.4 to 0.6 g/ml to create a mixture with a final concentration of α-cyclodextrin molecules in the mixture of between about 0.005 to about 0.008 g/ml;
c) mixing the solution of b) for a sufficient time to provide an inclusion step in which the hydrophobic polymers or derivatives thereof and cyclodextrin molecules obtain a pseudopolyrotaxane configuration in which the polymers or derivatives thereof are included in the cavity of each of α-cyclodextrin molecule in a skewered manner;
d) cooling the mixture of c) to room temperature;
e) evaporating the organic solvent away from mixture of d) to produce a dried product; and
f) washing the product of e) with water to remove excess α-cyclodextrin molecules.

25. The method of 24, wherein the hydrophobic polymer is PCL, the organic solvent is acetone, and the polar aprotic solvent is DMF.

26. The method of 25, further comprising:
g) dissolving the product of e) in a mixture of dichloromethane and DMSO to create a solution having a concentration between about 5% to about 15% w/v of polymer product; and
h) electrospinning the solution to create one or more nanofibers and allowing the fibers to dry.
